(19) United States
(12) Patent Application Publication
(10) Pub. No.: US 2023/0301924 A1
(43) Pub. Date: Sep. 28, 2023

(54) PHARMACEUTICAL FORMULATION

(71) Applicant: Janssen Pharmaceuticals, Inc., Titusville, NJ (US)

(72) Inventors: Donghua Zhu, Shanghai (CN); Kristof Leonard Kimpe, Beerse (BE); Kim Alda Verwaest, Lille (BE)

(21) Appl. No.: 18/035,259

(22) PCT Filed: Nov. 3, 2021
(86) PCT No.: PCT/CN2021/128520

§ 371 Date: May 3, 2023

(30) Foreign Application Priority Data
Nov. 4, 2020 (WO) PCT/CN2020/126597

(51) Int. Cl. A61K 9/48; A61K 31/404

(52) U.S. Cl. CPC A61K 9/4858; A61K 31/404

(57) ABSTRACT

The invention relates to pharmaceutical formulations comprising an active pharmaceutical ingredient, glyceryl palmitostearate, and D-α-tocopherol polyethylene glycol succinate (TPGS). Solid dosage forms comprising said pharmaceutical formulations, processes for preparing these and their use in methods of treatment are also described.

### FIELD OF THE INVENTION

The present invention relates to pharmaceutical formulations comprising an active pharmaceutical ingredient, glyceryl palmitostearate, and D-α-tocopherol polyethylene glycol succinate (TPGS), and solid dosage forms comprising said pharmaceutical formulations. The invention also relates to processes to prepare such pharmaceutical formulations and to the use of such pharmaceutical formulations for the treatment of a disease, syndrome, condition, or disorder.

### BACKGROUND OF THE INVENTION

Many active pharmaceutical ingredients (API) have properties such as hydrophobicity and instability leading to challenges in providing suitable pharmaceutical formulations.

Flaviviruses, which are transmitted by mosquitoes or ticks, cause life-threatening infections in man, such as encephalitis and hemorrhagic fever. Four distinct, but closely related serotypes of the flavivirus dengue (Dengue virus) are known. WO 2016/180696 discloses compounds and active pharmaceutical agents which show high potent activity against all four (4) serotypes of the Dengue virus.

There exists a need for improved pharmaceutical formulations of active pharmaceutical ingredients, such as the dengue viral replication inhibitors described in WO 2016/180696.

### SUMMARY OF THE INVENTION

The present invention is directed to a pharmaceutical formulation, comprising:
a) D-α-tocopherol polyethylene glycol succinate (TPGS);
b) an active pharmaceutical ingredient that is soluble in molten TPGS; and
c) glyceryl palmitostearate,

the D-α-tocopherol polyethylene glycol succinate (TPGS) may be TPGS 200, TPGS 300, TPGS 400, TPGS 1000, TPGS 1500, TPGS 2000 or TPGS 4000.

Embodiments of the invention include a pharmaceutical formulation as described herein, wherein the active pharmaceutical ingredient is a dengue viral replication inhibitor.

The invention also provides a solid dosage form comprising a pharmaceutical formulation as described herein.

In embodiments in which the active pharmaceutical ingredient is a dengue viral replication inhibitor, the invention provides methods for treating or preventing a disease, syndrome, condition, or disorder in a subject, including a mammal and/or human in which the disease, syndrome, condition, or disorder is a dengue viral infection, using pharmaceutical formulations and solid dosage forms described herein.

The invention is further directed to methods for inhibiting dengue virus replication in a mammal and/or human infected with dengue virus or at a risk of being infected with dengue virus.

The present invention is also directed to the use of such pharmaceutical formulations in the preparation of a medicament wherein the medicament is prepared for treating or preventing dengue viral infections.

In another embodiment, the present invention is directed to pharmaceutical formulations and solid dosage forms described herein for use in the treatment or prevention of dengue viral infections.

In certain embodiments, the present invention is directed to pharmaceutical formulations and solid dosage forms described herein for use in the inhibition of dengue virus replication in a mammal and/or human.

The invention also provides a process for preparing a pharmaceutical formulation as described herein, the process comprising the steps of:
a) forming a melt comprising glyceryl palmitostearate and D-α-tocopherol polyethylene glycol succinate (TPGS), wherein, in certain embodiments, the step of forming a melt comprises heating TPGS to a temperature above its melting point; said TPGS can be TPGS 200, TPGS 300, TPGS 400, TPGS 1000, TPGS 1500, TPGS 2000 or TPGS 4000.
   and
b) mixing the active pharmaceutical ingredient (while optionally stirring at the same temperature of step a)) until said active pharmaceutical ingredient dissolves;
   to provide a pharmaceutical formulation as described herein.

The invention also provides a process for preparing a solid dosage form described herein, the process comprising the steps of:
a) forming a melt comprising glyceryl palmitostearate and D-α-tocopherol polyethylene glycol succinate (TPGS), wherein, in certain embodiments, the step of forming a melt comprises heating TPGS to a temperature above its melting point; said TPGS can be TPGS 200, TPGS 300, TPGS 400, TPGS 1000, TPGS 1500, TPGS 2000 or TPGS 4000.
   and
b) mixing the active pharmaceutical ingredient (while optionally stirring at the same temperature of step a)) until said active pharmaceutical ingredient dissolves; and
c) filling a capsule, e.g., a hard capsule, with the melt;
   to provide a solid dosage form as described herein.

In certain embodiments, the invention provides a process for preparing a pharmaceutical formulation or a solid dosage form as described herein, the process comprising combining TPGS and the active pharmaceutical ingredient to form the pharmaceutical formulation or the solid dosage form.

### DETAILED DESCRIPTION OF THE INVENTION

The disclosure may be more fully appreciated by reference to the following description, including the following glossary of terms and the concluding examples. It is to be appreciated that certain features of the disclosed pharmaceutical formulations and methods which are, for clarity, described herein in the context of separate aspects, may also be provided in combination in a single aspect. Conversely, various features of the disclosed pharmaceutical formulations and methods that are, for brevity, described in the context of a single aspect, may also be provided separately or in any sub-combination.

Some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The terms “about” or “approximately” as used herein, when referring to a numerical value or range, allow for a degree of variability in the value or range, for example, within 10% (i.e., ±10%), within 5% (i.e., ±5%), or within 2.5% (i.e., ±2.5%) of a stated value or of a stated limit of a range.

Throughout the description and claims of this specification, the words “comprise” and “contain” and variations of the words, for example “comprising” and “comprises”, mean “including but not limited to”, and are not intended to (and do not) exclude other components.

The recitation of numerical ranges by endpoints includes all integer numbers and, where appropriate, fractions subsumed within that range (e.g. 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g. from 1.0 to 5.0 includes both 1.0 and 5.0). Any numerical range recited herein is intended to include all sub-ranges subsumed therein.

All references cited in the present specification are hereby incorporated by reference in their entirety. In particular, the teachings of all references herein specifically referred to are incorporated by reference.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art.

For use in medicine, cocrystals or salts of compounds of Formula (I) as disclosed herein refer to non-toxic “pharmaceutically acceptable salts”. “Pharmaceutically acceptable” may mean approved or approvable by a regulatory agency of the Federal or a state government or the corresponding agency in countries other than the United States, or that is listed in the U. S. Pharmacopoeia or other generally recognized pharmacopoeia for use in animals, and more particularly, in humans.

Other salts may, however, be useful in the preparation of compounds of Formula (I) or of their pharmaceutically acceptable salt forms thereof. Suitable pharmaceutically acceptable salts of compounds of Formula (I) include acid addition salts that can, for example, be formed by mixing a solution of the compound with a solution of a pharmaceutically acceptable acid such as, hydrochloric acid, sulfuric acid, fumaric acid, maleic acid, succinic acid, acetic acid, benzoic acid, citric acid, tartaric acid, carbonic acid or phosphoric acid. Furthermore, where the compounds of Formula (I) carry an acidic moiety, suitable pharmaceutically acceptable salts thereof may include alkali metal salts such as, sodium or potassium salts; alkaline earth metal salts such as, calcium or magnesium salts; and salts formed with suitable organic ligands such as, quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, and valerate.

Representative acids and bases that may be used in the preparation of pharmaceutically acceptable salts include acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine, and zinc hydroxide.

Where the compounds of Formula (I) have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the compounds may exist as polymorphs and as such are intended to be included in the present invention. In addition, some of the compounds may form solvates with water (i.e., hydrates) or common organic solvents, and such solvates are also intended to be encompassed within the scope of this invention. The skilled artisan will understand that the term compound as used herein, is meant to include solvated compounds of Formula (I).

Where the processes for the preparation of the compounds of Formula (I) give rise to mixture of stereoisomers, these isomers may be separated by conventional techniques such as, preparative chromatography. The compounds may be prepared in racemic form, or individual enantiomers may be prepared either by enantiospecific synthesis or by resolution. The compounds may, for example, be resolved into their component enantiomers by standard techniques such as, the formation of diastereomeric pairs by salt formation with an optically active acid such as, (−)-di-p-toluoyl-d-tartaric acid and/or (+)-di-p-toluoyl-l-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

In one embodiment of the pharmaceutical formulation of the present invention, the compound of Formula (I) is a compound comprising, consisting of, and/or consisting essentially of the (+)-enantiomer wherein said compound is substantially free from the (−)-isomer. In the present context, substantially free means less than about 25 wt %, preferably less than about 10 wt %, more preferably less than about 5 wt %, even more preferably less than about 2 wt % and even more preferably less than about 1 wt % of the (−)-isomer calculated as

$$\% ( + ) - enantiomer = \frac{( mass ( + ) - enantiomer )}{( mass ( + ) - enantiomer ) + ( mass ( - ) - enantiomer )} \times 100.$$

In another embodiment of the pharmaceutical formulation of the present invention, the compound of Formula (I) is a compound comprising, consisting of, and consisting essentially of the (−)-enantiomer wherein said compound is substantially free from the (+)-isomer. In the present context, substantially free from means less than about 25 wt %, preferably less than about 10 wt %, more preferably less than about 5 wt %, even more preferably less than about 2 wt % and even more preferably less than about 1 wt % of the (+)-isomer calculated as

$$\% ( - ) - enantiomer = \frac{( mass ( - ) - enantiomer )}{( mass ( + ) - enantiomer ) + ( mass ( - ) - enantiomer )} \times 100.$$

During any of the processes for preparation of the compounds of the various embodiments of the present invention, it may be necessary and/or desirable to protect sensitive or reactive groups on any of the molecules concerned. This may be achieved by means of conventional protecting groups such as those described in Protective Groups in Organic Chemistry, Second Edition, J. F. W. McOmie, Plenum Press, 1973; T. W. Greene & P. G. M. Wuts, Protective Groups in Organic Synthesis, John Wiley & Sons, 1991; and T. W. Greene & P. G. M. Wuts, Protective Groups in Organic Synthesis, Third Edition, John Wiley & Sons, 1999. The protecting groups may be removed at a convenient subsequent stage using methods known from the art.

The term “room temperature” (RT) refers to a temperature of from about 15° C. to about 30° C., in particular from about 20° C. to about 30° C. Preferably, room temperature is a temperature of about 25° C.

An average molecular weight may, for example, refer to a number average or weight average molecular weight. Average molecular weight may, for example, be measured using gel permeation chromatography.

The term “subject” refers to an animal, preferably a mammal, most preferably a human, who has been the object of treatment, observation or experiment.

The term “therapeutically effective amount” refers to an amount of an active compound or active pharmaceutical ingredient which elicits the biological or medicinal response in a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease.

The terms “active compound”, “active ingredient” and “active pharmaceutical ingredient” are herein used interchangeably.

In embodiments in which the active pharmaceutical ingredient is a dengue viral replication inhibitor, the term “therapeutically effective amount” may refer to the amount of a formulation of the present invention that, when administered to a subject, is effective to at least partially alleviate, inhibit, prevent, and/or ameliorate a condition, or a disorder or a disease caused by a Dengue virus in said subject.

As used herein, the term “Dengue virus” refers to the single positive-stranded RNA virus of the family Flaviviridae; four distinct, but closely related serotypes of the flavivirus dengue are known, so-called DENV1, -2, -3, and -4. Flaviviruses, which are transmitted by mosquitoes or ticks, cause life-threatening infections in man, such as encephalitis and hemorrhagic fever.

As used herein, the term “dengue viral replication inhibitor” refers to an agent that inhibits or reduces at least one condition, symptom, disorder, and/or disease caused by a Dengue virus.

As used herein, unless otherwise noted, the term “affect” or “affected” (when referring to a disease, syndrome, condition or disorder that is affected by the inhibition of a Dengue virus replication) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physical parameter including those which may not be discernible by the patient. In a further embodiment, “treat”, “treating”, or “treatment” refers to modulating the disease, condition, syndrome or disorder either physically (e.g. stabilization of a discernible symptom), physiologically, (e.g. stabilization of a physical parameter), or both. In yet another embodiment, “treat”, “treating”, or “treatment” refers to preventing or delaying the onset or development or progression of the disease, condition, syndrome or disorder.

Preferred statements (features) and embodiments of the polymer compositions, pharmaceutical compositions, articles, uses and process of this invention are set herein below. Each statement and embodiment of the invention so defined may be combined with any other statement and/or embodiment unless clearly indicated to the contrary. In particular, any feature indicated as being preferred or advantageous may be combined with any other feature or features or statements indicated as being preferred or advantageous. Hereto, the present invention is in particular captured by any one or any combination of one or more of the below numbered aspects and embodiments, with any other statement and/or embodiments.

1. A pharmaceutical formulation, comprising:
a) D-α-tocopherol polyethylene glycol 1000 succinate (TPGS);
b) an active pharmaceutical ingredient that is soluble in molten TPGS; and
c) glyceryl palmitostearate.
   2. The pharmaceutical formulation of statement 1, wherein the pharmaceutical formulation is a solid formulation.
   3. The pharmaceutical formulation of any one of statements 1 or 2, wherein the TPGS and the glyceryl palmitostearate are present in said formulation in a ratio of from 95:5 w/w to 70:30 w/w, preferably from 95:5 w/w to 80:20 w/w; more preferably from 90:10 w/w to 75:25 w/w, more preferably from 85:15 w/w to 75:25 w/w, more preferably from 85:15 w/w to 80:20 w/w.
   4. The pharmaceutical formulation of any one of statements 1 to 3, wherein the formulation further comprises an antioxidant.
   5. The pharmaceutical formulation of any one of statements 1 to 4, wherein the formulation comprises from 0.001 wt % to 2.0 wt % of the antioxidant relative to the total weight of the formulation; preferably from 0.005 wt % to 1.0 wt % of the antioxidant; preferably 0.005 wt % to 0.5 wt % of the antioxidant.
   6. The pharmaceutical formulation of statements 4 or 5, wherein the antioxidant is selected from ascorbic palmitate, tocopherol (vitamin E), thiodipropionic acid, lipoic acid, hydroquinone, phytic acid, monothioglycerol, sodium thioglycolate, thioglycol, beta carotene, butylated hydroxyanisole (BHA), butylated hydroxytoluene (BHT), cysteine, cysteine hydrochloride, propyl gallate (PG), sodium metabisulfite, ascorbyl stearate, potassium metabisulfite, disodium EDTA (ethylenediamine tetraacetic acid; also known as disodium edentate), EDTA, erythorbic acid, ethoxyquin, glutathione, gum guaiac, lecithin, propyl gallate, TBHQ (tert butyl hydroxyquinone), tartaric acid, citric acid, citric acid monohydrate, methane sulfonic acid, methionine, sodium metabisulfite, sodium thiosulfate, sodium sulphite, and any combinations thereof.
   7. The pharmaceutical formulation of statements 4-6, wherein the antioxidant is selected from ascorbic palmitate, tocopherol (vitamin E), lipoic acid, hydroquinone, monothioglycerol, thioglycol, beta carotene, butylated hydroxyanisole (BHA), butylated hydroxytoluene (BHT), propyl gallate (PG), ascorbyl stearate, ethoxyquin, propyl gallate, TBHQ (tert butyl hydroxyquinone), and any combinations thereof.
   8. The pharmaceutical formulation of any one of statements 1 to 7, wherein the formulation comprises from 0.1 wt % to 40 wt %, preferably from 1 wt % to 30 wt %, preferably from 2.5 wt % to 25 wt % of the active pharmaceutical ingredient relative to the total weight of the formulation.
   9. The pharmaceutical formulation of any one of statements 1 to 7, wherein the formulation comprises from 2.0 wt % to 35 wt % of the active pharmaceutical ingredient relative to the total weight of the formulation.
   10. The pharmaceutical formulation of any one of statements 1 to 9, wherein the active pharmaceutical ingredient is a dengue viral replication inhibitor.
   11. The pharmaceutical formulation of any one of statements 1 to 10, wherein the active pharmaceutical ingredient is a compound of Formula (I)

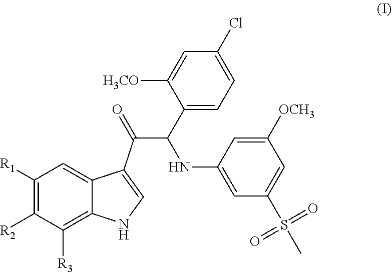

a stereo-isomeric form, a pharmaceutically acceptable salt, solvate or polymorph thereof said compound is selected from the group wherein:
R₁ is H, R₂ is F and R₃ is H or CH₃,
R₁ is H, CH₃ or F, R₂ is OCH₃ and R₃ is H and
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H and
R₁ is OCF₃, R₂ is H and R₃ is CH₃.
   12. The pharmaceutical formulation according to statement 11, wherein the compound of Formula (I) is:

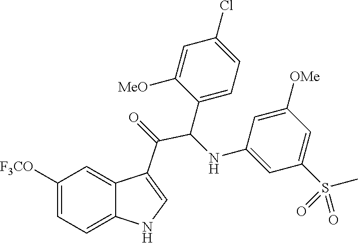 [SMILES: COc1cc(NC(C(=O)c2c[nH]c3ccc(OC(F)(F)F)cc23)c2ccc(Cl)cc2OC)cc(S(C)(=O)=O)c1]

or a stereo-isomeric form, a pharmaceutically acceptable salt, solvate or polymorph thereof.
13. A solid dosage form comprising the pharmaceutical formulation of any one of statements 1 to 12.
14. The solid dosage form of statement 13, wherein the dosage form is an oral dosage form.
15. The solid dosage form of statements 13 or 14, wherein the dosage form comprises a capsule encapsulating the pharmaceutical formulation.
16. The solid dosage form of statement 15, wherein the capsule is a hard gelatin capsule.
17. The solid dosage form of any one of statements 13-16, wherein the pharmaceutical formulation comprises from 0.5 to 1000 mg of the active pharmaceutical ingredient (API); preferably the formulation comprises from 1 to 1000 mg of the API; preferably the formulation comprises from 1 to 900 mg of the API; preferably the formulation comprises from 2 to 500 mg of the API.
18. The solid dosage form of any one of statements 13 to 17, wherein the formulation comprises 2, 10, 50, 100 or 200 mg of the API; preferably the active pharmaceutical ingredient is:

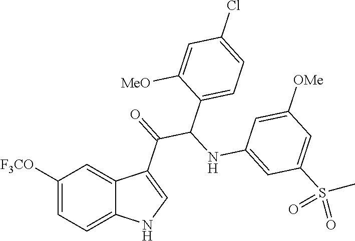 [SMILES: COc1cc(NC(C(=O)c2c[nH]c3ccc(OC(F)(F)F)cc23)c2ccc(Cl)cc2OC)cc(S(C)(=O)=O)c1]

a pharmaceutically acceptable salt, solvate or polymorph thereof.
19. A method of treating or preventing of dengue viral infections, comprising administering to a subject in need thereof a therapeutically effective amount of a pharmaceutical formulation of any one of statements 1 to 12 or of a solid dosage form of any one of statements 13-18.
20. The method of statement 19, comprising inhibiting dengue virus replication in a mammal and/or human infected with dengue virus or at a risk of being infected with dengue virus.
21. The use of a pharmaceutical formulation of any one of statements 1 to 12 or of a solid dosage form of any one of statements 13-18 for the preparation of a medicament for treating or preventing of dengue viral infections.
22. A pharmaceutical formulation of any one of statements 1 to 12 or a solid dosage form of any one of statements 13-18 for use in a method for treating or preventing of dengue viral infections.
23. A process for preparing a pharmaceutical formulation according to any one of statements 1 to 12, comprising the steps of:
a) forming a melt comprising glyceryl palmitostearate and TPGS, wherein the step of forming a melt comprises heating the TPGS to a temperature above its melting point; and
b) mixing the active pharmaceutical ingredient and stirring at the same temperature of step a) until said active pharmaceutical ingredient dissolves;
   to provide a pharmaceutical formulation according to any one of statements 1 to 12.
   24. The process of statement 23, wherein an antioxidant is added to the melt, preferably in step a).
   25. The process of any one of statements 23-24, wherein the active pharmaceutical ingredient is a compound of Formula (I)

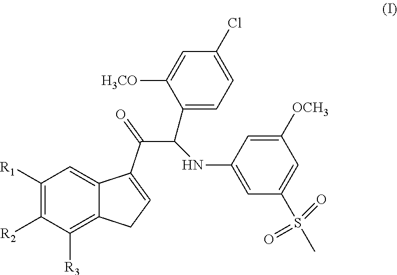

a stereo-isomeric form, a pharmaceutically acceptable salt, solvate or polymorph thereof; said compound is selected from the group wherein:
R₁ is H, R₂ is F and R₃ is H or CH₃,
R₁ is H, CH₃ or F, R₂ is OCH₃ and R₃ is H and
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H and
R₁ is OCF₃, R₂ is H and R₃ is CH₃.
   25. The process of any one of statements 23-24, wherein the active pharmaceutical ingredient is:

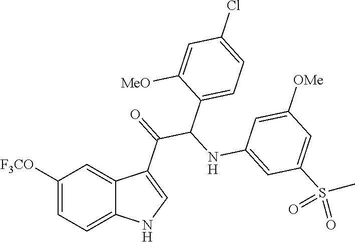 [SMILES: COc1cc(NC(C(=O)c2c[nH]c3ccc(OC(F)(F)F)cc23)c2ccc(Cl)cc2OC)cc(S(C)(=O)=O)c1]

a pharmaceutically acceptable salt, solvate or polymorph thereof.

### Pharmaceutical Formulations

The invention provides a pharmaceutical formulation, comprising:
a) D-α-tocopherol polyethylene glycol 1000 succinate (TPGS);
b) an active pharmaceutical ingredient that is soluble in molten TPGS; and
c) glyceryl palmitostearate.

In an embodiment, the invention provides a pharmaceutical formulation, comprising:
a) D-α-tocopherol polyethylene glycol 1000 succinate (TPGS);
b) an active pharmaceutical ingredient (API) which is

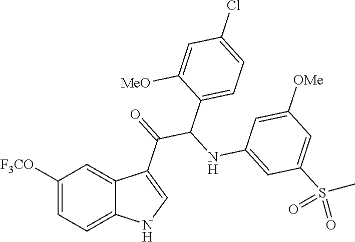 [SMILES: COc1cc(NC(C(=O)c2c[nH]c3ccc(OC(F)(F)F)cc23)c2ccc(Cl)cc2OC)cc(S(C)(=O)=O)c1]

a pharmaceutically acceptable salt, solvate or polymorph thereof and
c) glyceryl palmitostearate.

In an embodiment, the invention provides a pharmaceutical formulation, comprising:
a) D-α-tocopherol polyethylene glycol 1000 succinate (TPGS);
b) an active pharmaceutical ingredient (API);
c) glyceryl palmitostearate; and
d) an antioxidant.

In an embodiment, the invention provides a pharmaceutical formulation, comprising:
a) D-α-tocopherol polyethylene glycol 1000 succinate (TPGS);
b) an active pharmaceutical ingredient (API) which is

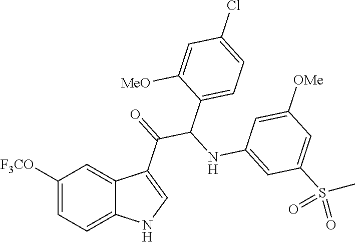 [SMILES: COc1cc(NC(C(=O)c2c[nH]c3ccc(OC(F)(F)F)cc23)c2ccc(Cl)cc2OC)cc(S(C)(=O)=O)c1]

a pharmaceutically acceptable salt, solvate or polymorph thereof;
c) glyceryl palmitostearate; and
d) an antioxidant.

In an embodiment, the API is soluble in the D-α-tocopherol polyethylene glycol 1000 succinate (TPGS) molten at a temperature of 5° C. to 35° C. above the upper limit melting point of said TPGS. Preferably molten at a temperature of 7° C. to 35° C.; preferably at a temperature of 5° C. to 30° C.; preferably at a temperature of 7° C. to 30° C. above the upper limit melting point of said TPGS. The upper limit melting point of TPGS at atmospheric pressure ranges from 35° C. to 45° C., preferably from 36° C. to 43° C., more preferably from 37° C. to 41° C. It is to be understood that in any embodiment of the invention, the D-α-tocopherol polyethylene glycol 1000 succinate (TPGS) may be replaced by other TPGS such as, without limitations, TPGS 200, TPGS 300, TPGS 400, TPGS 1500, TPGS 2000 or TPGS 4000, or a combination thereof.

The pharmaceutical formulation of the invention may comprise at most 50 wt %, at most 45 wt %, at most 40 wt %, at most 35 wt %, or at most 30 wt % of the active pharmaceutical ingredient (API) relative to the total weight of the formulation. The pharmaceutical formulation may comprise at least 0.1 wt %, at least 1 wt %, at least 5 wt %, at least 10 wt %, of the API relative to the total weight of the formulation. The pharmaceutical formulation may comprise from 0.1 wt % to 40 wt %, from 1 wt % to 30 wt %, or from 5 wt % to 25 wt % of the API relative to the total weight of the formulation.

The pharmaceutical formulation of the invention may contain from 0.1 mg to 3000 mg of the API, from 1 mg to 2000 mg of the API, from 5 mg to 1000 mg of the API, from 10 mg to 500 mg of the API, from 20 mg to 400 mg of the API, from 30 mg to 300 mg of the API, from 40 mg to 200 mg of the API, from 50 mg to 100 mg of the API, from 60 mg to 90 mg of the API or from 70 mg to 90 mg of the API or any particular amount or range comprised therein. The therapeutically effective amount for said API will vary as will the diseases, syndromes, conditions, and disorders being treated.

The pharmaceutical formulation of the invention may comprise at least 20 wt %, at least 30 wt %, at least 40 wt %, at least 50 wt %, at least 60 wt %, or at least 65 wt % D-α-Tocopherol polyethylene glycol, preferably D-α-Tocopherol polyethylene glycol 1000 succinate relative to the total weight of the formulation. The pharmaceutical formulation may comprise from 60 wt % to 95 wt %, from 65 wt % to 90 wt %, from 65 wt % to 85 wt %, of D-α-Tocopherol polyethylene glycol 1000 succinate relative to the total weight of the formulation. D-α-Tocopherol polyethylene glycol 1000 succinate (also known as TPGS or Vitamin E TPGS) is formed by the esterification of Vitamin E succinate with polyethylene glycol 1000. D-α-Tocopherol polyethylene glycol 1000 succinate exist at room temperature as waxy solids. Its melting point at atmospheric pressure is from 37-41° C.

The pharmaceutical formulation of the invention may be a solid dispersion. In particular, the pharmaceutical formulation may be a solid solution. Solid solutions are discussed in Leuner & Dressman, Eur. J Pharm. Biopharm., 50, 2000, 47-60, which is incorporated herein by reference.

The pharmaceutical formulation of the invention also comprises glyceryl palmitostearate. Glyceryl palmitostearate is a mixture of mono-, di-, and tri-glyceryl esters of palmitic and stearic acids made from glycerin, palmitic acid, and stearic acid. Glyceryl palmitostearate has a higher melting point than D-α-Tocopherol polyethylene glycol 1000 succinate.

The pharmaceutical formulation of the invention may comprise at most 20 wt % of the glyceryl palmitostearate relative to the total weight of the formulation. The pharmaceutical formulation may comprise at least 0.1 wt % of glyceryl palmitostearate relative to the total weight of the formulation. The pharmaceutical formulation may comprise from 1 wt % to 15 wt % glyceryl palmitostearate relative to the total weight of the formulation. The pharmaceutical formulation may comprise from 1 wt %, from 5 wt % or from 10 wt % of glyceryl palmitostearate.

In some embodiments the pharmaceutical formulation of the invention comprises TPGS and glyceryl palmitostearate present in a ratio of from 95:5 w/w to 80:20 w/w; preferably a ratio of from 95:5 w/w to 85:15 w/w; preferably a ratio of from 94:6 w/w to 80:20 w/w; preferably a ratio of from 93:7 w/w to 85:15 w/w. In some embodiments the pharmaceutical formulation of the invention comprises TPGS and glyceryl palmitostearate present in a ratio of from 95:5 w/w to 70:30 w/w, preferably from 90:10 w/w to 75:25 w/w, more preferably from 85:15 w/w to 80:20 w/w.

The pharmaceutical formulation of the invention optionally comprises an antioxidant. The antioxidant may be selected from ascorbic palmitate, tocopherol (vitamin E), thiodipropionic acid, lipoic acid, hydroquinone, phytic acid, monothioglycerol, sodium thioglycolate, thioglycol, vitamin E acetate, beta carotene, butylated hydroxyanisole (BHA), butylated hydroxytoluene (BHT), cysteine, cysteine hydrochloride, propyl gallate (PG), sodium metabisulfite, ascorbyl stearate, potassium metabisulfite, disodium EDTA (ethylenediamine tetraacetic acid; also known as disodium edentate), EDTA, erythorbic acid, ethoxyquin, glutathione, gum guaiac, lecithin, TBHQ (tert butyl hydroxyquinone), tartaric acid, citric acid, citric acid monohydrate, methane sulfonic acid, methionine, sodium metabisulfite, sodium thiosulfate, sodium sulphite, and a combination thereof.

The antioxidant may be selected from ascorbic palmitate, tocopherol (vitamin E), lipoic acid, hydroquinone, monothioglycerol, thioglycol, beta carotene, butylated hydroxyanisole (BHA), butylated hydroxytoluene (BHT), propyl gallate (PG), ascorbyl stearate, ethoxyquin, propyl gallate, TBHQ (tert butyl hydroxyquinone), and a combination thereof. The antioxidant may be ascorbic palmitate, tocopherol (vitamin E), propyl gallate or any combination thereof. The antioxidant may be ascorbic palmitate.

The pharmaceutical formulation of the invention may comprise from 0.001 wt % to 2 wt % of antioxidant relative to the total weight of the formulation. The pharmaceutical formulation may comprise from 0.001 wt % to 1 wt % of antioxidant relative to the total weight of the formulation. The pharmaceutical formulation may comprise from 0.01 wt % to 2 wt % of antioxidant relative to the total weight of the formulation. The pharmaceutical formulation may comprise from 0.01 wt % to 1 wt % of antioxidant relative to the total weight of the formulation. The pharmaceutical formulation may comprise from 0.01 wt % to 0.5 wt % of antioxidant relative to the total weight of the formulation.

The pharmaceutical formulation of the invention may further comprise one or more pharmaceutically acceptable excipients, as described in more detail herein. Pharmaceutically acceptable excipients include, but are not limited to, disintegrants, binders, diluents, lubricants, stabilizers, osmotic agents, colorants, plasticizers, coatings and the like.

More particularly, suitable pharmaceutical excipients comprise one or more of the following: (i) diluents such as lactose, mannitol, microcrystalline cellulose, dicalcium phosphate, maltodextrin, starch and the like; (ii) binders such as polyvinylpyrrolidone (such as povidone), methylcellulose, hydroxypropyl cellulose, hydroxypropyl methylcellulose (such as METHOCEL® E-5), and the like; (iii) disintegrants such as sodium starch glycolate, croscamellose sodium, crospovidone, L-HPC (low substituted hydroxypropylcellulose), pregelatinized starch, maize starch and the like; (iv) wetting agents such as surfactants, such as sodium lauryl stearate, docusate sodium, polysorbate 20, polysorbate 80 and the like; (v) lubricants such as magnesium stearate, sodium stearyl fumarate, stearic acid, talc, and the like; (vi) flow promoters or glidants such as colloidal silicon dioxide, talc and the like; and other excipients known to be useful in the preparation of pharmaceutical formulations; (vii) stabilizers such as myristic acid, palmitic acid, stearic acid, cetyl alcohol, cetostearyl alcohol, stearylalcohol, glyceryl distearate, glycerol monostearate, glyceryl dibehenate, hard fat or any combination thereof. Additional suitable pharmaceutical excipients and their properties may be found in texts such as Handbook of Pharmaceutical Excipients, Edited by R. C. Rowe, P. J. Sheskey & P. J. Weller, Sixth Edition (Published by Pharmaceutical Press, a Division of Royal Pharmaceutical Society of Great Britain).

Fillers or diluents for use in the pharmaceutical formulations of the present invention include fillers or diluents typically used in the formulation of pharmaceuticals. Examples of fillers or diluents for use in accordance with the present invention include, but are not limited to, sugars such as lactose, dextrose, glucose, sucrose, cellulose, starches and carbohydrate derivatives, polysaccharides (including dextrates and maltodextrin), polyols (including mannitol, xylitol, and sorbitol), cyclodextrins, calcium carbonates, magnesium carbonates, microcrystalline cellulose, combinations thereof, and the like. In certain preferred embodiments the filler or diluent is lactose, microcrystalline cellulose, or combination thereof. Several types of microcrystalline cellulose are suitable for use in the formulations described herein, for example, microcrystalline cellulose selected from the group consisting of Avicel® types: PH101, PH102, PH103, PH105, PH112, PH113, PH200, PH301, and other types of microcrystalline cellulose, such as silicified microcrystalline cellulose. Several types of lactose are suitable for use in the formulations described herein, for example, lactose selected from the group consisting of anhydrous lactose, lactose monohydrate, lactose fast flo, directly compressible anhydrous lactose, and modified lactose monohydrate.

Binders for use in the pharmaceutical formulations of the present invention include binders commonly used in the formulation of pharmaceuticals. Examples of binders for use in accordance with the present invention include but are not limited to cellulose derivatives (including hydroxypropyl cellulose, hydroxypropyl methylcellulose, methylcellulose, and sodium carboxymethyl cellulose), glycol, sucrose, dextrose, corn syrup, polysaccharides (including acacia, targacanth, guar, alginates and starch), corn starch, pregelatinized starch, modified corn starch, gelatin, polyvinylpyrrolidone, polyethyleneglycol, combinations thereof and the like.

Disintegrants for use in the pharmaceutical formulations of the present invention include disintegrants commonly used in the formulation of pharmaceuticals. Examples of disintegrants for use in accordance with the present invention include but are not limited to starches, and crosslinked starches, celluloses and polymers, combinations thereof and the like. Representative disintegrants include microcrystalline cellulose, croscarmellose sodium, alginic acid, sodium alginate, crosprovidone, cellulose, agar and related gums, sodium starch glycolate, corn starch, potato starch, sodiumstarch glycolate, Veegum HV, methylcellulose, L-HPC (low substituted hydroxypropylcellulose), agar, bentonite, sodium carboxymethylcellulose, calcium carboxymethylcellulose, carboxymethylcellulose, alginic acid, guar gum, maize starch, pregelatinized starch, combinations thereof, and the like.

Lubricants, glidants or anti-tacking agents for use in the pharmaceutical formulations of the present invention include lubricants, glidants and anti-tacking agents commonly used in the formulation of pharmaceuticals. Examples for use in accordance with the present invention include but are not limited to magnesium carbonate, magnesium laurylsulphate, calcium silicate, talc, fumed silicon dioxide, combinations thereof, and the like. Other useful lubricants include but are not limited to magnesium stearate, calcium stearate, stearic acid, sodium stearyl fumarate, sodium lauryl sulphate, magnesium lauryl sulphate, sodium benzoate, colloidal silicon dioxide, magnesium aluminometasilicate (such as Neusilin®), magnesium oxide, magnesium silicate, mineral oil, hydrogenated vegetable oils, waxes, glyceryl behenate, and combinations thereof, and the like.

Surfactants for use in the pharmaceutical formulations of the present invention include surfactants commonly used in the formulation of pharmaceuticals. Examples of surfactants for use in accordance with the present invention include but are not limited to zwitterionic, ionic- and nonionic surfactants or wetting agents commonly used in the formulation of pharmaceuticals, such as ethoxylated castor oil, polyglycolyzed glycerides, acetylated monoglycerides, sorbitan fatty acid esters, poloxamers (e.g. Pluronic®), polyethylene glycol (15)-hydroxystearate (e.g. Solutol®), polyoxyethylene sorbitan fatty acid esters, polyoxyethylene derivatives, monoglycerides or ethoxylated derivatives thereof, diglycerides or polyoxyethylene derivatives thereof, dioctyl sulfosuccinate sodium salt (sodium docusate), sodium laurylsulfate (SLS), cholic acid or derivatives thereof, lecithins, phospholipids, combinations thereof, and the like. Non-ionic surfactants may have an HLB (hydrophile-lipophile balance) value higher than 10.

The pharmaceutical formulations disclosed herein can further comprise one or more flow regulators (or glidants). Flow regulators may be present in powders or granules and are admixed in order to increase their flowability of the formulation during manufacture, particularly in the preparation of tablets produced by pressing powders or granules. Flow regulators which can be employed include, but are not limited to, highly disperse silicon dioxide (Aerosil®) or dried starch.

Tablet dosage forms may further comprise a coating. Suitable coatings are film-forming polymers, such as, for example, those from the group of the cellulose derivatives (such as HPC (hydroxypropylcellulose), HPMC (hydroxypropoxymethylcellulose), MC (methylcellulose), HPMCAS (hydroxypropoxymethylcelluclose acetate succinate)), dextrins, starches, natural gums, such as, for example, gum arabic, xanthans, alginates, polyvinyl alcohol, polymethacrylates and derivatives thereof, such as, for example, Eudragit®, which may be applied to the tablet as solutions or suspensions by means of the various pharmaceutical conventional methods, such as, for example, film coating. The coating is typically applied as a solution/suspension which, in addition to any film-forming polymer present, may further comprise one or more adjuvants, such as hydrophilisers, plasticisers, surfactants, dyes and white pigments, such as, for example, titanium dioxide.

One skilled in the art will readily recognize that the appropriate pharmaceutically acceptable excipients are selected such that they are compatible with other excipients and do not bind or interact with the active pharmaceutical ingredient or cause degradation of the active ingredient or of the pharmaceutical formulation.

The pharmaceutical formulation can be obtained by
a) forming a melt comprising glyceryl palmitostearate and D-α-tocopherol polyethylene glycol 1000 succinate (TPGS), wherein the step of forming a melt comprises heating the TPGS to a temperature above its melting point; and
b) mixing the active pharmaceutical ingredient and stirring at the same temperature of step a) until said active pharmaceutical ingredient dissolves.

It will be appreciated that any of the above description relating to components of the pharmaceutical formulation may apply to any of the other aspects and embodiments of the invention.

### Active Pharmaceutical Ingredient

Suitable active pharmaceutical ingredients are those which exert a pharmacological, immunological or metabolic action with a view to restoring, correcting or modifying physiological functions or to make a medical diagnosis. Non-limiting examples thereof include analgesic and anti-inflammatory drugs; anti-arrhythmic drugs; antibacterial and antiprotozoal agents; anti-coagulants; antidepressants; anti-diabetic drugs; anti-epileptic drugs; antifungal agents; antihistamines; anti-hypertensive drugs; anti-muscarinic agents; antineoplastic agents and antimetabolites; anti-migraine drugs; anti-Parkinsonian drugs; antipsychotic, hypnotic and sedating agents; anti-stroke agents; antitussive; antivirals; beta-adrenoceptor blocking; cardiac inotropic agents; corticosteroids; disinfectants; diuretics; enzymes; essential oils; gastro-intestinal agents; lipid regulating agents; local anaesthetics; opioid analgesics; parasympathomimetics and anti-dementia drugs; sex hormones; stimulating agents and vasodilators.

The invention provides a pharmaceutical formulation, comprising:
a) D-α-tocopherol polyethylene glycol 1000 succinate (TPGS);
b) an active pharmaceutical ingredient that is soluble in molten TPGS; and
c) glyceryl palmitostearate.

In an embodiment, the API is soluble in TPGS which is molten at a temperature of from 5° C. to 35° C. above the melting point of said TPGS. In an embodiment, the API is soluble in TPGS which is molten at a temperature of from 5° C. to 35° C. above the upper limit of the melting point of said TPGS. The solubility may be measured at a temperature above the melting point of the TPGS or may be measured using hot stage microscopy.

Preferably, the API is sufficiently soluble in the molten TPGS to enable a therapeutically effective dose of the API to be administered in a formulation of the invention. Preferably, the solubility of the API in the formulation is sufficient to ensure long term physical stability (up to 2 years, 3 years or 4 years) in a dissolved state at the desired concentration in the formulation. The concentration of API may be as high as deemed necessary to limit the size of the particular dosage form (e.g. capsule size and number) to be taken by a patient in order to reach the therapeutically effective dose. For example, if a capsule size of at most size 00 (dosage form volume=1 mL) is recommended to allow ease of swallowability and if the estimated targeted therapeutic dose is up to 1 g, 5 capsules of a 200 mg/dosage form per day would be desired for a patient to reach the therapeutically effective targeted dose. Therefore, in this example, the API would have a solubility of at least 200 mg/mL in the formulation. Lower solubility would represent an increase in the number of capsules in order to reach the estimated therapeutically effective dose.

The API may have a solubility of at least 1, 5, 10, 20, 50, 100, 200, 220, 250, 290, 300, 320, 350 mg/g in TPGS at a temperature of 45° C. The API may have a solubility of at least 1, 5, 10, 20, 50, 100, 200, 220, 250, 290, 300, 320, 350 mg/g in TPGS at a temperature of 50° C. The API may have a solubility of at least 1, 5, 10, 20, 50, 100, 200, mg/g in TPGS at a temperature of 53° C. The API may have a solubility of at least 1, 5, 10, 20, 50, 100, 200, 220, 250, 290, 300, 320, 350 mg/g in TPGS at a temperature of 55° C.

Solubility may be measured using a classical shake-flask determination (within a range using visual assessment or by chromatographic analysis of the filtrate in case of filtration/supernatant in case of centrifugation). This method is typically used for determination at 50° C. which is above TPGS melting point, i.e. in a liquid matrix. Solubility may be measured using hot stage microscopy or differential scanning microscopy (DSC). This method is typically used for determination of solubility in a solid matrix, e.g. at room temperature.

In an embodiment, the API has poor solubility in water. In an embodiment, the API has a solubility of at most 50, 20, 10, 1, 0.1, 0.01 or 0.001 mg/g in water (measured by shake flask at room temperature, using chromatographic analysis (UPLC)). Solubility may be measured e.g. at 25° C. or 50° C. using the shake-flask method. The API may be defined as sparingly soluble (from 30 to 100 parts water for 1 part API), slightly soluble (from 100 to 1000 parts water for 1 part API), very slightly soluble (from 1000 to 10,000 parts water for 1 part API), or practically insoluble (more than 10,000 parts water for 1 part API) in water, as defined by The Pharmacopeia of the United States of America, in the chapter “General notices and Requirements” (Page information USP42-NF37 2S-9081; Section 5.30 Description and Solubility).

In particular, the API is in amorphous form or dissolved state (i.e. molecular dispersion) in the pharmaceutical formulation.

In a preferred embodiment, the active pharmaceutical ingredient (API) is a dengue viral replication inhibitor. For example, embodiments of the invention include a pharmaceutical formulation as described herein, wherein the active pharmaceutical ingredient is a compound of Formula (I)

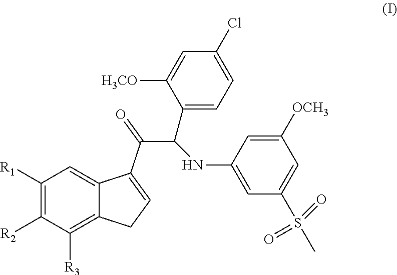

a stereo-isomeric form, a pharmaceutically acceptable salt, solvate or polymorph thereof; said compound is selected from the group wherein:
R₁ is H, R₂ is F and R₃ is H or CH₃,
R₁ is H, CH₃ or F, R₂ is OCH₃ and R₃ is H and
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H and
R₁ is OCF₃, R₂ is H and R₃ is CH₃.

Additional embodiments of the invention include pharmaceutical formulations as described herein, wherein the active pharmaceutical ingredient is a compound of Formula (I) selected from the group consisting of:

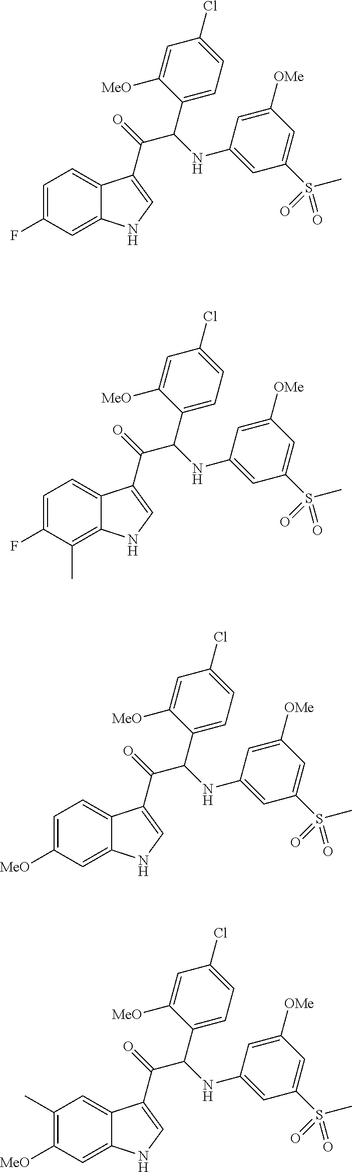 [SMILES: COc1cc(NC(C(=O)c2c[nH]c3cc(F)ccc23)c2ccc(Cl)cc2OC)cc(S(C)(=O)=O)c1 ; COc1cc(NC(C(=O)c2c[nH]c3c(C)c(F)ccc23)c2ccc(Cl)cc2OC)cc(S(C)(=O)=O)c1 ; COc1cc(NC(C(=O)c2c[nH]c3cc(OC)ccc23)c2ccc(Cl)cc2OC)cc(S(C)(=O)=O)c1 ; COc1cc(NC(C(=O)c2c[nH]c3cc(OC)c(C)cc23)c2ccc(Cl)cc2OC)cc(S(C)(=O)=O)c1]

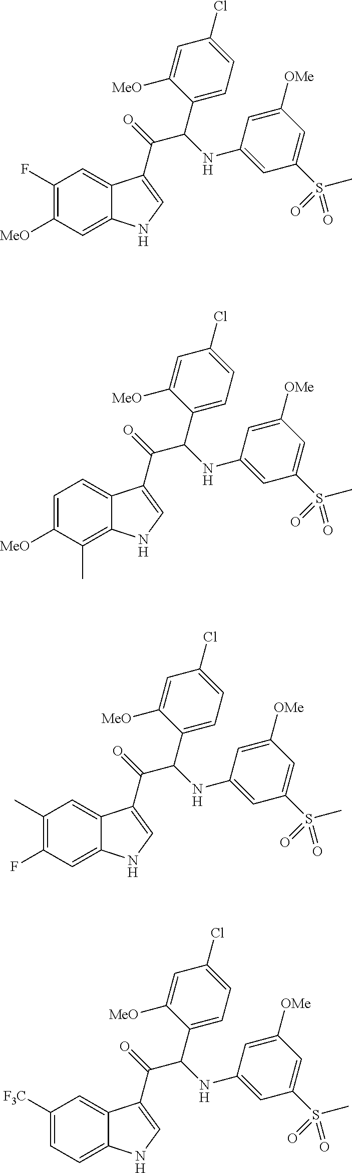 [SMILES: COc1cc(NC(C(=O)c2c[nH]c3cc(OC)c(F)cc23)c2ccc(Cl)cc2OC)cc(S(C)(=O)=O)c1 ; COc1cc(NC(C(=O)c2c[nH]c3c(C)c(OC)ccc23)c2ccc(Cl)cc2OC)cc(S(C)(=O)=O)c1 ; COc1cc(NC(C(=O)c2c[nH]c3cc(F)c(C)cc23)c2ccc(Cl)cc2OC)cc(S(C)(=O)=O)c1 ; COc1cc(NC(C(=O)c2c[nH]c3ccc(C(F)(F)F)cc23)c2ccc(Cl)cc2OC)cc(S(C)(=O)=O)c1]

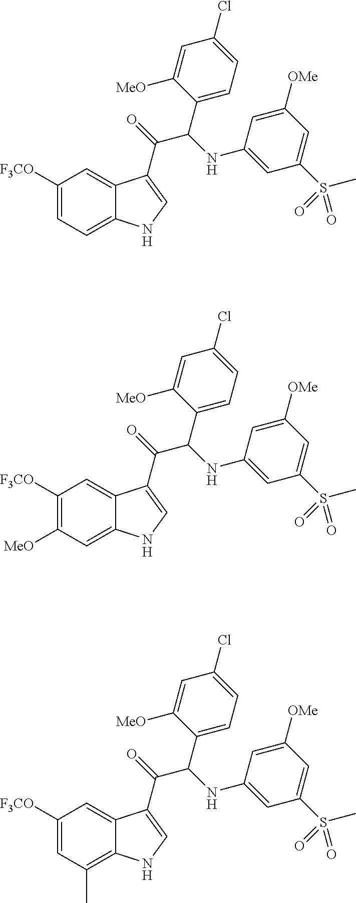 [SMILES: COc1cc(NC(C(=O)c2c[nH]c3ccc(OC(F)(F)F)cc23)c2ccc(Cl)cc2OC)cc(S(C)(=O)=O)c1 ; COc1cc(NC(C(=O)c2c[nH]c3cc(OC)c(OC(F)(F)F)cc23)c2ccc(Cl)cc2OC)cc(S(C)(=O)=O)c1 ; COc1cc(NC(C(=O)c2c[nH]c3c(C)cc(OC(F)(F)F)cc23)c2ccc(Cl)cc2OC)cc(S(C)(=O)=O)c1]

or its stereo-isomeric form, a pharmaceutically acceptable salt, solvate or polymorph thereof.

In particular, the API is a compound of Formula (I), or an enantiomer, diastereomer or pharmaceutically acceptable salt form thereof.

In particular, the API is a compound of Formula (I), or an enantiomer, diastereomer or pharmaceutically acceptable salt form thereof, in amorphous state or dissolved state (i.e. molecular dispersion).

In particular, the API used as starting material in the process to prepare a pharmaceutical formulation as described herein, is a compound of Formula (I), or an enantiomer, diastereomer, solvate, or a pharmaceutically acceptable salt form thereof; while the API in the final pharmaceutical formulation or solid dosage form as defined herein is a compound of Formula (I), or an enantiomer, diastereomer, or pharmaceutically acceptable salt form thereof, in amorphous form or dissolved state.

In a preferred embodiment, the compound of Formula (I) is

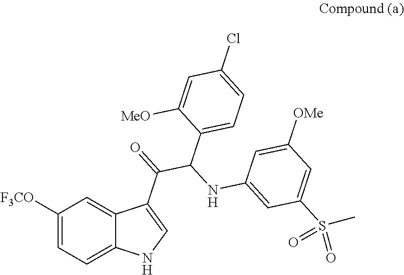 [SMILES: COc1cc(NC(C(=O)c2c[nH]c3ccc(OC(F)(F)F)cc23)c2ccc(Cl)cc2OC)cc(S(C)(=O)=O)c1]

The API may be Compound (a) or a solvate or pharmaceutically acceptable salt form thereof. The API may be Compound (a) or a pharmaceutically acceptable salt form thereof. The API may be Compound (a) in a solvated form, for example as a monohydrate. Preferably the API is Compound (a). Preferably the API is the (S)-enantiomer of Compound (a). Preferably the API is Compound (a) in anhydrous form. Preferably the API is Compound (a) in amorphous form. Preferably the API is Compound (a) or a pharmaceutically acceptable salt form thereof in amorphous form or dissolved state. Preferably the API is Compound (a) in amorphous form or dissolved state. Preferably the API is the (S)-enantiomer of Compound (a) in amorphous form. Preferably the API is the (S)-enantiomer of Compound (a) in anhydrous form.

In particular, the API used as starting material in the process to prepare a pharmaceutical formulation as described herein, is Compound (a) in a solvated form, or a pharmaceutically acceptable salt form thereof; while the API in the final pharmaceutical formulation or solid dosage form is Compound (a) or a pharmaceutically acceptable salt form thereof in amorphous form or dissolved state (i.e. molecular dispersion).

Compounds of formula (I) can be synthesized according to the procedures disclosed in WO 2016/180696, which is incorporated herein by reference in its entirety.

It will be appreciated that any of the above description relating to active pharmaceutical ingredients may apply to any embodiment of the pharmaceutical formulations, solid dosage forms, processes, uses, and methods of treatment described herein. For example, any reference to a dengue viral replication inhibitor may refer to a compound of formula (I), or a stereo-isomeric form, a pharmaceutically acceptable salt, solvate, cocrystal or polymorph thereof.

In a particular embodiment, the API in the pharmaceutical formulation as described herein is Compound (a), or a stereo-isomeric form, a pharmaceutically acceptable salt, solvate or polymorph thereof. In a particular embodiment, the API in the pharmaceutical formulation as described herein is Compound (a).

In a particular embodiment, the API in the pharmaceutical formulation as described herein is a dengue viral replication inhibitor in amorphous form or dissolved state. In a particular embodiment, the API in the pharmaceutical formulation as described herein is Compound (a) or a pharmaceutically acceptable salt form thereof, in amorphous form or dissolved state. In a particular embodiment, the API in the pharmaceutical formulation as described herein is Compound (a) in amorphous form or dissolved state.

### Solid Dosage Form

The invention also provides a solid dosage form comprising a pharmaceutical formulation as described herein.

The solid dosage form may comprise a capsule encapsulating the pharmaceutical formulation. The capsule may be a hard capsule (also called hard shell capsule) or a soft shell capsule. The hard capsule may be a hypromellose (HMPC) capsule (e.g. Vegicap®, VCaps®, VCaps® Plus, or Quali-V®) or a gelatin capsule (e.g. ConiSnap®, Licaps®, or Quali-G™). The hard capsule encapsulates a unit dose of the formulation.

The dosage form may be an oral dosage form (e.g. a capsule for oral administration). Alternatively, the dosage form may be an enteral dosage form.

Typically a hard capsule (e.g. a hard gelatin capsule) comprises two part capsule shells, one of which is first filled with the formulation, the other of which is connected to the first in a telescoping manner to close the capsule. The two part capsule shells are typically adhered together by applying solvent (e.g. water or aqueous ethanol) to the interface between the two shells to create a bond between the two part shells. This differs to the manufacturing processes used for soft gelatin capsules, wherein the formulation is enclosed between half-capsule shells as the soft capsule is formed.

Hard gelatin (hard gel) capsules are generally used for solid, semi-solid, and some compatible liquid formulations, while soft gelatin (soft gel) capsules are generally used for liquid formulations. Hard gel capsules may be preferable for some formulations. Soft gel capsules contain a higher percentage of water than hard gel capsules. This can result in problems when the soft gel contains liquid formulations of poorly water soluble APIs. Water leaching from the soft gel capsule into the formulation may lower the maximum drug loading for that capsule. Higher maximum drug load may be achieved for a poorly water soluble drug when using a hard gel capsule compared to a soft gel capsule.

Additionally, hard gel capsules can more easily be used in blister packs than soft gel capsules, as there is a lower risk of bursting the capsule when forcing it through the foil of the blister.

The solid dosage form may alternatively be a tablet.

The solid dosage form as described herein (e.g. a capsule, e.g. a hard gelatin capsule) may contain from 0.1 mg to 3000 mg of the API, from 1 mg to 2000 mg of the API, from 5 mg to 1000 mg of the API, from 10 mg to 500 mg of the API, from 20 mg to 400 mg of the API, from 30 mg to 300 mg of the API, from 40 mg to 200 mg of the API, from 50 mg to 100 mg of the API, from 60 mg to 90 mg of the API or from 70 mg to 90 mg of the API or any particular amount or range comprised therein. The therapeutically effective amount for said API will vary as will the diseases, syndromes, conditions, and disorders being treated.

The solid dosage form as described herein (e.g. a capsule, e.g. a hard gelatin capsule) may contain from 0.5 mg to 1000 mg of the API. In some embodiments the solid dosage form may comprise from 0.5 mg to 1000 mg, for example from 1.0 mg to 500 mg, for example from 2.0 mg to 400 mg, for example from 5.0 mg to 300 mg, for example from 10 mg to 200 mg of API; preferably the API is

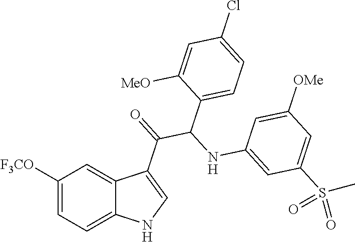 [SMILES: COc1cc(NC(C(=O)c2c[nH]c3ccc(OC(F)(F)F)cc23)c2ccc(Cl)cc2OC)cc(S(C)(=O)=O)c1]

(Compound (a)), a pharmaceutically acceptable salt, solvate or polymorph thereof. The solid dosage form may comprise 2, 10, 50, 100 or 200 mg of Compound (a) a pharmaceutically acceptable salt, solvate or polymorph thereof. The solid dosage form may comprise 2, 10, 50 or 200 mg of Compound (a) a pharmaceutically acceptable salt, solvate or polymorph thereof.

In a particular embodiment, the solid dosage form is a capsule comprising
a) D-α-tocopherol polyethylene glycol 1000 succinate (TPGS);
b) an active pharmaceutical ingredient that is soluble in molten TPGS; and
c) glyceryl palmitostearate.

In a particular embodiment, the solid dosage form is a capsule comprising
a) D-α-tocopherol polyethylene glycol 1000 succinate (TPGS);
b) an active pharmaceutical ingredient that is soluble in molten TPGS;
c) glyceryl palmitostearate; and
d) an antioxidant.

In a particular embodiment, the solid dosage form is a tablet comprising
a) D-α-tocopherol polyethylene glycol 1000 succinate (TPGS);
b) an active pharmaceutical ingredient that is soluble in molten TPGS; and
c) glyceryl palmitostearate.

In a particular embodiment, the solid dosage form is a tablet comprising
a) D-α-tocopherol polyethylene glycol 1000 succinate (TPGS);
b) an active pharmaceutical ingredient that is soluble in molten TPGS;
c) glyceryl palmitostearate; and
d) an antioxidant.

In a particular embodiment, the solid dosage form is a capsule consisting of
a) D-α-tocopherol polyethylene glycol 1000 succinate (TPGS);
b) an active pharmaceutical ingredient that is soluble in molten TPGS; and
c) glyceryl palmitostearate.

In a particular embodiment, the solid dosage form is a capsule consisting of
a) D-α-tocopherol polyethylene glycol 1000 succinate (TPGS);
b) an active pharmaceutical ingredient that is soluble in molten TPGS;
c) glyceryl palmitostearate; and
d) an antioxidant.

In a particular embodiment, the solid dosage form is a tablet consisting of
a) D-α-tocopherol polyethylene glycol 1000 succinate (TPGS);
b) an active pharmaceutical ingredient that is soluble in molten TPGS; and
c) glyceryl palmitostearate.

In a particular embodiment, the solid dosage form is a tablet consisting of
a) D-α-tocopherol polyethylene glycol 1000 succinate (TPGS);
b) an active pharmaceutical ingredient that is soluble in molten TPGS;
c) glyceryl palmitostearate; and
d) an antioxidant.

In a particular embodiment, the solid dosage form is a capsule consisting essentially of
a) D-α-tocopherol polyethylene glycol 1000 succinate (TPGS);
b) an active pharmaceutical ingredient that is soluble in molten TPGS; and
c) glyceryl palmitostearate.

In a particular embodiment, the solid dosage form is a capsule consisting essentially of
a) D-α-tocopherol polyethylene glycol 1000 succinate (TPGS);
b) an active pharmaceutical ingredient that is soluble in molten TPGS;
c) glyceryl palmitostearate; and
d) an antioxidant.

In a particular embodiment, the solid dosage form is a tablet consisting essentially of
a) D-α-tocopherol polyethylene glycol 1000 succinate (TPGS);
b) an active pharmaceutical ingredient that is soluble in molten TPGS; and
c) glyceryl palmitostearate.

In a particular embodiment, the solid dosage form is a tablet consisting essentially of
a) D-α-tocopherol polyethylene glycol 1000 succinate (TPGS);
b) an active pharmaceutical ingredient that is soluble in molten TPGS;
c) glyceryl palmitostearate; and
d) an antioxidant.

In a particular embodiment, the solid dosage form is a capsule comprising a pharmaceutical formulation of the present invention.

In a particular embodiment, the solid dosage form is a tablet comprising a pharmaceutical formulation of the present invention.

In an embodiment, the solid dosage form comprises a pharmaceutical formulation, wherein the pharmaceutical formulation comprises 2, 10, 50, 100 or 200 mg of the API; preferably the API is:

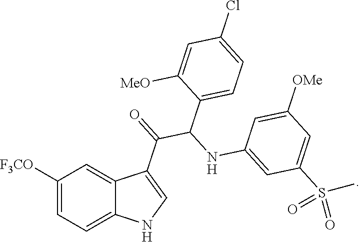 [SMILES: COc1cc(NC(C(=O)c2c[nH]c3ccc(OC(F)(F)F)cc23)c2ccc(Cl)cc2OC)cc(S(C)(=O)=O)c1]

In an embodiment, the solid dosage form comprises a pharmaceutical formulation, wherein the pharmaceutical formulation comprises 2, 10, 50, 100 or 200 mg of the API; preferably the API is

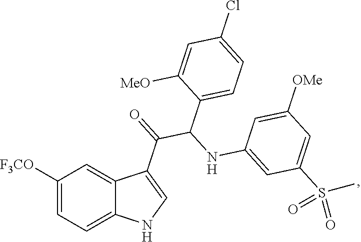 [SMILES: COc1cc(NC(C(=O)c2c[nH]c3ccc(OC(F)(F)F)cc23)c2ccc(Cl)cc2OC)cc(S(C)(=O)=O)c1]

or a pharmaceutically acceptable salt form thereof.

The capsule of the solid dosage form may be a hydroxypropylmethylcellulose (HPMC) capsule.

For enteric administration, a solid dosage form is in particular provided in the form of tablets containing at least 0.5 mg, at least 1 mg, at least 10 mg, at least 20 mg, at least 30 mg, at least 40 mg, at least 50 mg, at least 60 mg, at least 70 mg, at least 80 mg, at least 90 mg, at least 100 mg, at least 110 mg, at least 120 mg, at least 130 mg, at least 140 mg, at least 150 mg, at least 160 mg, at least 170 mg, at least 180 mg, at least 190 mg, at least 200 mg, at least 210 mg, at least 220 mg, at least 230 mg, at least 240 mg, at least 250 mg, at least 260 mg, at least 270 mg, at least 280 mg, at least 290 mg, at least 300 mg, at least 310 mg, at least 320 mg, at least 330 mg, at least 340 mg, at least 350 mg, at least 360 mg, at least 370 mg, at least 380 mg, at least 390 mg, at least 400 mg, at least 410 mg, at least 420 mg, at least 430 mg, at least 440 mg, at least 450 mg, at least 460 mg, at least 470 mg, at least 480 mg, at least 490 mg, at least 500 mg, at least 510 mg, at least 520 mg, at least 530 mg, at least 540 mg, at least 550 mg, at least 560 mg, at least 570 mg, at least 580 mg, at least 590 mg, at least 600 mg, at least 610 mg, at least 620 mg, at least 630 mg, at least 640 mg, at least 650 mg, at least 660 mg, at least 670 mg, at least 680 mg, at least 690 mg, at least 700 mg, at least 710 mg, at least 720 mg, at least 730 mg, at least 740 mg, at least 750 mg, at least 760 mg, at least 770 mg, at least 780 mg, at least 790 mg, at least 800 mg, at least 810 mg, at least 820 mg, at least 830 mg at least 840 mg, at least 850 mg, at least 860 mg, at least 870 mg, at least 880 mg, at least 890 mg at least 900 mg, at least 910 mg, at least 920 mg, at least 930 mg, at least 940 mg, at least 950 mg at least 960 mg, at least 970 mg, at least 980 mg, at least 990 mg, at least 1000 mg, at least 1100 mg at least 1200 mg, at least 1210 mg, at least 1220 mg, at least 1230 mg, at least 1240 mg, at least 1250 mg at least 1260 mg, at least 1270 mg, at least 1280 mg, at least 1290 mg, at least 1300 mg, at least 1410 mg at least 1320 mg, at least 1330 mg, at least 1340 mg, at least 1350 mg, at least 1360 mg, at least 1370 mg at least 1390 mg, at least 1400 mg of API or any value or range comprised in the aforementioned values; in particular from 25 mg to 500 mg of API. Said enteric administration is preferably oral administration.

For enteric administration, a solid dosage form is in particular provided in the form of capsules containing at least 0.5 mg, at least 1 mg, at least 10 mg, at least 20 mg, at least 30 mg, at least 40 mg, at least 50 mg, at least 60 mg, at least 70 mg, at least 80 mg, at least 90 mg, at least 100 mg, at least 110 mg, at least 120 mg, at least 130 mg, at least 140 mg, at least 150 mg, at least 160 mg, at least 170 mg, at least 180 mg, at least 190 mg, at least 200 mg, at least 210 mg, at least 220 mg, at least 230 mg, at least 240 mg, at least 250 mg, at least 260 mg, at least 270 mg, at least 280 mg, at least 290 mg, at least 300 mg, at least 310 mg, at least 320 mg, at least 330 mg, at least 340 mg, at least 350 mg, at least 360 mg, at least 370 mg, at least 380 mg, at least 390 mg, at least 400 mg, at least 410 mg, at least 420 mg, at least 430 mg, at least 440 mg, at least 450 mg, at least 460 mg, at least 470 mg, at least 480 mg, at least 490 mg, at least 500 mg, at least 510 mg, at least 520 mg, at least 530 mg, at least 540 mg, at least 550 mg, at least 560 mg, at least 570 mg, at least 580 mg, at least 590 mg, at least 600 mg, at least 610 mg, at least 620 mg, at least 630 mg, at least 640 mg, at least 650 mg, at least 660 mg, at least 670 mg, at least 680 mg, at least 690 mg, at least 700 mg, at least 710 mg, at least 720 mg, at least 730 mg, at least 740 mg, at least 750 mg, at least 760 mg, at least 770 mg, at least 780 mg, at least 790 mg, at least 800 mg, at least 810 mg, at least 820 mg, at least 830 mg at least 840 mg, at least 850 mg, at least 860 mg, at least 870 mg, at least 880 mg, at least 890 mg at least 900 mg, at least 910 mg, at least 920 mg, at least 930 mg, at least 940 mg, at least 950 mg at least 960 mg, at least 970 mg, at least 980 mg, at least 990 mg, at least 1000 mg, at least 1100 mg at least 1200 mg, at least 1210 mg, at least 1220 mg, at least 1230 mg, at least 1240 mg, at least 1250 mg at least 1260 mg, at least 1270 mg, at least 1280 mg, at least 1290 mg, at least 1300 mg, at least 1410 mg at least 1320 mg, at least 1330 mg, at least 1340 mg, at least 1350 mg, at least 1360 mg, at least 1370 mg at least 1390 mg, at least 1400 mg of API or any value or range comprised in the aforementioned values; in particular from 25 mg to 500 mg of API. Said enteric administration is preferably oral administration.

Advantageously, the API may be administered in a single daily dose, or the total daily dosage may be administered in divided doses of two, three, four or five daily. The daily dose may be maintained unchanged throughout all days or some days of a treatment or prevention period. Said daily dose may change throughout the days of a treatment or prevention period such as it increases and/or decreases during the days of said treatment or prevention period. For instance, said daily dose may be unchanged for the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 days or more (or any single value or range comprised thereon) followed by a lower and/or a higher daily dose for the remaining days of the treatment or prevention period. Said remaining days of the treatment or prevention period can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days or more (or any single value or range comprised thereon).

In certain embodiments, the API may be administered at one dose for a first duration (e.g., a loading phase) and at a second dose for a second duration (e.g., maintenance phase). The loading phase may include administration of any of the dosages described herein (e.g., from about 10 mg to about 1000 mg, from about 25 mg to about 800 mg, or from about 50 mg to about 400 mg). The first duration of administration in the loading phase may be for any of the time periods contemplated herein (e.g., from about 1 day to about 40 days, from about 3 days to about 20 days, or from about 5 days to about 10 days). The maintenance phase may include administration of any of the dosages described herein (e.g., from about 10 mg to about 1000 mg, from about 25 mg to about 800 mg, or from about 50 mg to about 400 mg). The second duration of administration in the maintenance phase may be for any of the periods contemplated herein (e.g., from about 1 day to about 60 days, from about 5 days to about 45 days, or from about 10 days to about 30 days).

Optimal dosages of the pharmaceutical formulation to be administered may be readily determined and will vary with the particular compound used, the mode of administration, the strength of the preparation, and the advancement of the disease, syndrome, condition or disorder. In addition, factors associated with the particular subject being treated, including subject gender, age, weight, diet and time of administration, will result in the need to adjust the dose to achieve an appropriate therapeutic level and desired therapeutic effect. The above dosages are thus exemplary of the average case. There can be, of course, individual instances wherein higher or lower dosage ranges are merited, and such are within the scope of this invention.

The invention also provides a process for preparing a pharmaceutical formulation, as described herein. The process may comprise the steps of
a) forming a melt comprising glyceryl palmitostearate and D-α-tocopherol polyethylene glycol 1000 succinate (TPGS), wherein the step of forming a melt comprises heating the TPGS to a temperature above its melting point; and
b) mixing the active pharmaceutical ingredient and stirring at the same temperature of step a) until said active pharmaceutical ingredient dissolves;
   to provide a pharmaceutical formulation as described herein.

The invention also provides a process for preparing a solid dosage form, as described herein. The process may comprise the steps of:
a) forming a melt comprising glyceryl palmitostearate and D-α-tocopherol polyethylene glycol 1000 succinate (TPGS), wherein the step of forming a melt comprises heating the TPGS to a temperature above its melting point;
b) mixing the active pharmaceutical ingredient and stirring at the same temperature of step a) until said active pharmaceutical ingredient dissolves; and
c) filling a hard capsule (e.g. a gelatin or hypromellose capsule) with the melt;
to provide a solid dosage form as described herein.

In an embodiment, the melt is formed under an inert atmosphere. In another embodiment, the melt is formed under nitrogen.

In an embodiment, the melt further comprises an antioxidant, for example ascorbic palmitate. The melt may further comprise one or more pharmaceutically acceptable excipients, as described herein.

The step of forming a melt comprises heating TPGS to a temperature above its melting point. The TPGS may be heated to a temperature of at least 5, 10, 15, 20, 25, 30 or 35° C. above its melting point. In particular the TPGS may be heated to a temperature of at least 5, 10, 15, 20, 25, 30 or 35° C. above the upper limit of its melting point. The TPGS may be heated to a temperature of up to 75° C., for example from 46° C. to 75° C.; preferably from 50° C. to 70° C. The TPGS may be heated to a temperature of 55, 60, 65 or 70° C. In certain embodiments, TPGS is melted at a temperature below its melting point when combined with suitable excipients.

The step of forming a melt may comprise adding the API to molten TPGS and glyceryl palmitostearate. The step of forming a melt comprises heating the TPGS to a temperature above its melting point.

In particular, the melt is a semi-liquid melt or liquid melt.

In particular, the melt is a liquid melt.

The hard capsule may be filled using a capsule filling machine hopper. The machine hopper may be preheated to a temperature above the melting point of the TPGS, wherein the temperature is as described above.

The process may further comprise the step of packaging the capsules in bottles (e.g. HDPE bottles), followed by induction sealing. Alternatively, the process may further comprise the step of sealing the capsules in blister packs.

This process may be advantageous compared to traditional processes for manufacturing solid dosage forms. The molten formulation can be easily dispensed into a capsule. This reduces the number of steps usually associated with the manufacture of solid formulations.

A solid dosage form of the invention may be prepared using a spray congealing process, comprising the steps of: a) forming a melt comprising TPGS, wherein the step of forming a melt comprises heating the TPGS to a temperature above its melting point; b) mixing the active pharmaceutical ingredient and stirring at the same temperature of step a) until said active pharmaceutical ingredient dissolves; and c) atomizing the melt into cold nitrogen. The atomized melt may be compressed into tablets.

A solid dosage form of the invention may be prepared by a screw granulation process, for example using twin-screw extruders that continuously mix and granulate the glyceryl palmitostearate, TPGS, and active pharmaceutical ingredient (and optionally maltodextrin). The resulting granules may be compressed into tablets.

A solid dosage form of the invention may be prepared by loading a melt of glyceryl palmitostearate, TPGS and active pharmaceutical ingredient onto a porous clay-type particle, such as magnesium aluminometasilicate (e.g. Neusilin®) or silica, to obtain a powder which may be compressed into tablets.

It will be appreciated that any of the above discussion relating to solid dosage forms and processes for their preparation may apply to any embodiments of solid dosage forms, processes and treatments described herein.

### Methods of Treatment

The pharmaceutical formulations described herein may be administered in any of the foregoing dosage forms and regimens or by means of those dosage forms and regimens established in the art whenever use of the pharmaceutical formulation is required for a subject in need thereof.

The pharmaceutical formulations and dosage forms of the present invention are useful in methods for treating, ameliorating and/or preventing a disease, a syndrome, a condition or a disorder in a subject in need thereof. Such methods comprise, consist of and/or consist essentially of administering to a subject, including an animal, a mammal, and a human in need of such treatment, amelioration and/or prevention, a therapeutically effective amount of a formulation or dosage form described herein. In embodiments in which the active pharmaceutical ingredient is a dengue viral replication inhibitor, the pharmaceutical formulations and dosage forms of the present invention are useful in methods for treating, ameliorating and/or preventing a disease, a syndrome, a condition that is affected by the inhibition of dengue viral replication.

One embodiment of the present invention is directed to a method of treating a dengue viral infection in a subject in need thereof, including an animal, a mammal, and a human in need of such treatment, comprising administering to the subject a therapeutically effective amount of a pharmaceutical formulation or dosage form described herein.

In certain embodiments, the blood plasma level of the API is at a level, e.g., for the duration of the treatment regimen (for treatment or for prevention), that is in the range of about 5 ng/ml to about 10,000 ng/ml, about 10 ng/ml to about 8,000 ng/ml, about 15 ng/ml to about 6,500 ng/ml, about 20 ng/ml to about 5,000 ng/ml, about 25 ng/ml to about 4,500 ng/ml, about 30 ng/ml to about 3,000 ng/ml, about 40 ng/ml to about 2,000 ng/ml, or about 50 ng/ml to about 1,000 ng/ml, or any single value or sub-range therein. In certain embodiments, the maximum blood plasma level of the API is up to about 10,000 ng/ml, up to about 8,000 ng/ml, up to about 6,500 ng/ml, up to about 4,500 ng/ml, up to about 3,000 ng/ml, up to about 2,000 ng/ml, up to about 1,000 ng/ml or any single value or sub-range therein. In certain embodiments, the minimum blood plasma level of the API, e.g., for the duration of the treatment regimen (for treatment or for prevention), is at least about 5 ng/ml, at least about 10 ng/ml, at least about 15 ng/ml, at least about 20 ng/ml, at least about 25 ng/ml, at least about 30 ng/ml, at least about 40 ng/ml, at least about 50 ng/ml, or any single value or sub-range therein. The blood plasma levels referred to here may be obtained with any of the doses and/or dosing regimens described herein.

In another embodiment of the present invention, the pharmaceutical formulations described herein may be employed in combination with one or more other medicinal agents, more particularly with other antiviral agents.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All possible combinations of the above-indicated embodiments are considered to be embraced within the scope of this invention.

Reference is now made to the following examples, which illustrate the invention in a non-limiting fashion.

### GENERAL SYNTHETIC METHODS

Representative compounds of the present invention can be synthesized in accordance with the general synthetic methods described below and illustrated in the schemes and examples that follow. Since the schemes are an illustration, the invention should not be construed as being limited by the chemical reactions and conditions described in the schemes and examples. Compounds analogous to the target compounds of these examples can be made according to similar routes. The disclosed compounds are useful as active pharmaceutical ingredients as described herein. The various starting materials used in the schemes and examples are commercially available or may be prepared by methods well within the skill of persons versed in the art.

### Compounds of Formula I

The synthesis of compounds of general formula I can be performed as outlined in Scheme 1. 2-(4-Chloro-2-methoxyphenyl)acetic acid (II) can be converted to the corresponding 2-(4-chloro-2-methoxyphenyl)acetyl chloride (III) with a chlorination reagent like for example thionyl chloride. The Friedel-Crafts reaction of the acid chloride III with a substituted indole of general formula IV can be performed using a Lewis acid reagent like for example Et₂AlCl or TiCl₄ in a suitable solvent like for example CH₂Cl₂ or 1,2-dichloroethane, and under suitable reaction conditions that typically (but not exclusively) involve cooling, to provide the 3-acylated indole of general formula V. The introduction of an aniline moiety in alpha position to the carbonyl moiety of the compounds of general formula V can be accomplished by a reaction sequence that involves for example bromination of V with a reagent like for example phenyltrimethylammonium tribromide in a suitable solvent like for example tetrahydrofuran (THF), to provide the compounds of general formula VI, and subsequent reaction of the compounds of general formula VI with 3-methoxy-5-(methylsulfonyl)aniline (VII) in a suitable solvent like for example CH₃CN, and typically using a base like for example triethylamine (TEA) or N,N-Diisopropylethylamine (DIPEA), to provide the compounds of general formula I as racemic mixtures. Chiral separation of the compounds of general formula I can be performed by for example chiral chromatography to provide the Enantiomers A and B of general formula I.

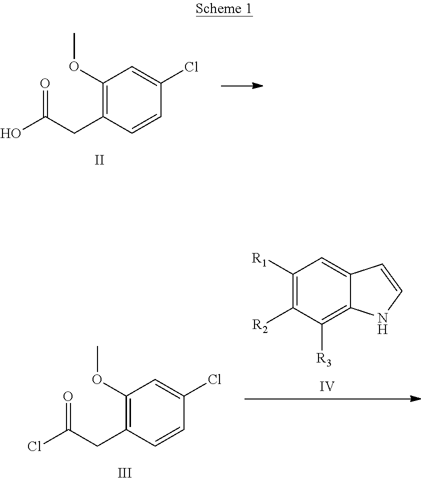

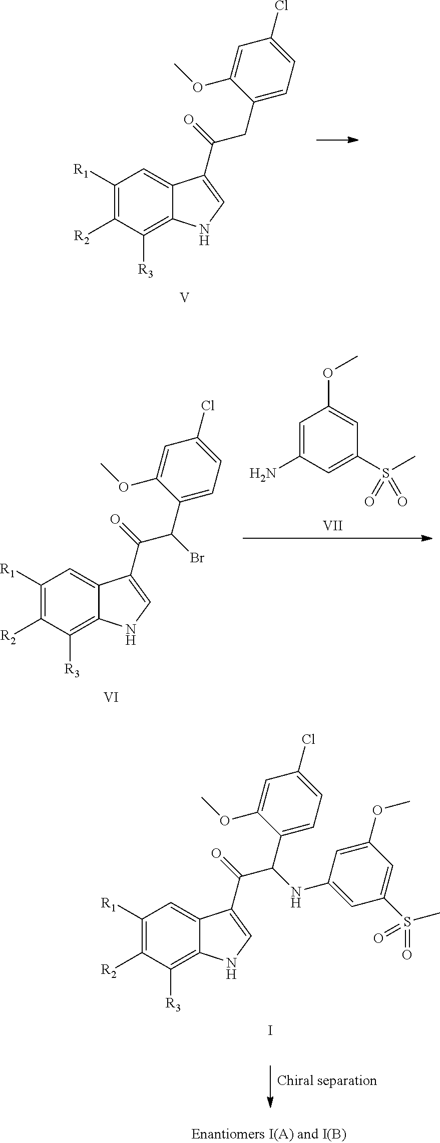

In some cases, the synthesis of the intermediate of general formula V via the Friedel-Crafts synthesis approach, benefits from the presence of a protecting group (PG) at the indole-N during the Friedel-Crafts reaction step, as outlined in Scheme 2. To this end, the substituted indole of general formula IV can be converted first to an N-protected intermediate of general formula VIII, such as for example an N-Tosylated intermediate of general formula VIII (PG=Ts), using a reagent like for example tosyl chloride, in the presence of a base like for example sodium hydride. The Friedel-Crafts reaction of the substituted indole of general formula IV with acid chloride III can be performed using a Lewis acid reagent like for example Et₂AlCl or TiCl₄ in a suitable solvent like for example CH₂Cl₂ or 1,2-dichloroethane, and under suitable reaction conditions that typically (but not exclusively) involve cooling, to provide the 3-acylated N-protected indole of general formula IX. Removal of the indole-N protecting group PG of the intermediate of general formula IX can be accomplished with a reagent like for example LiOH (for PG=Ts) in a solvent mixture like for example THF/water and at a suitable reaction temperature, to provide the 3-acylated indole of general formula V.

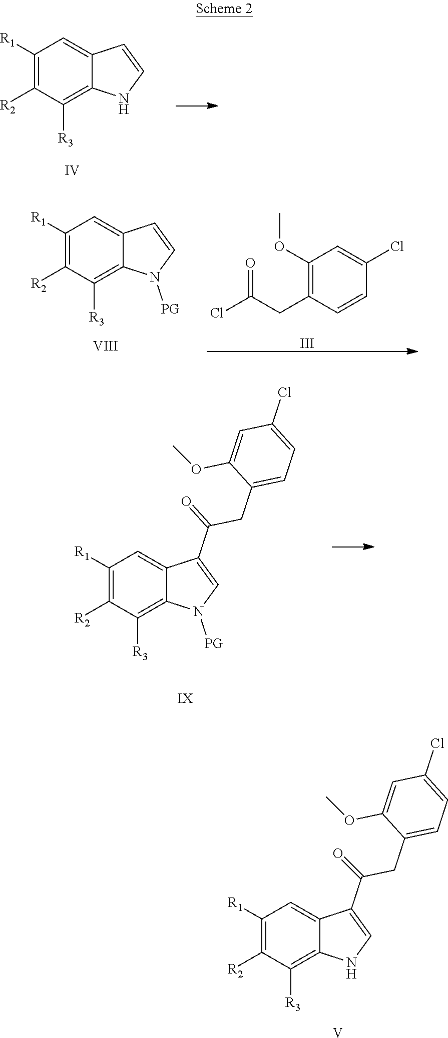

As an alternative approach, the intermediate of general formula V can also be prepared as outlined in Scheme 3: The N-Boc-protected substituted indole-3-carbaldehyde of general formula X can be converted to the corresponding Strecker-type of intermediate of general formula XI by reaction with morpholine in the presence of reagents like for example sodium cyanide and sodium bisulfite and in a suitable solvent like for example a mixture of water and a water-mixable organic solvent like for example dioxane. Alkylation of the compound of general formula XI with 4-chloro-2-methoxy-benzylchloride can be accomplished in the presence of a base like for example potassium hexamethyldisilazane and in a suitable solvent like for example dimethylformamide (DMF) to provide the compound of general formula XII. Submission of the compound of general formula XII to a suitable aqueous acidic hydrolytic condition like for example by treatment with an aqueous hydrochloric acid solution at elevated temperature, provides the intermediate of general formula V.

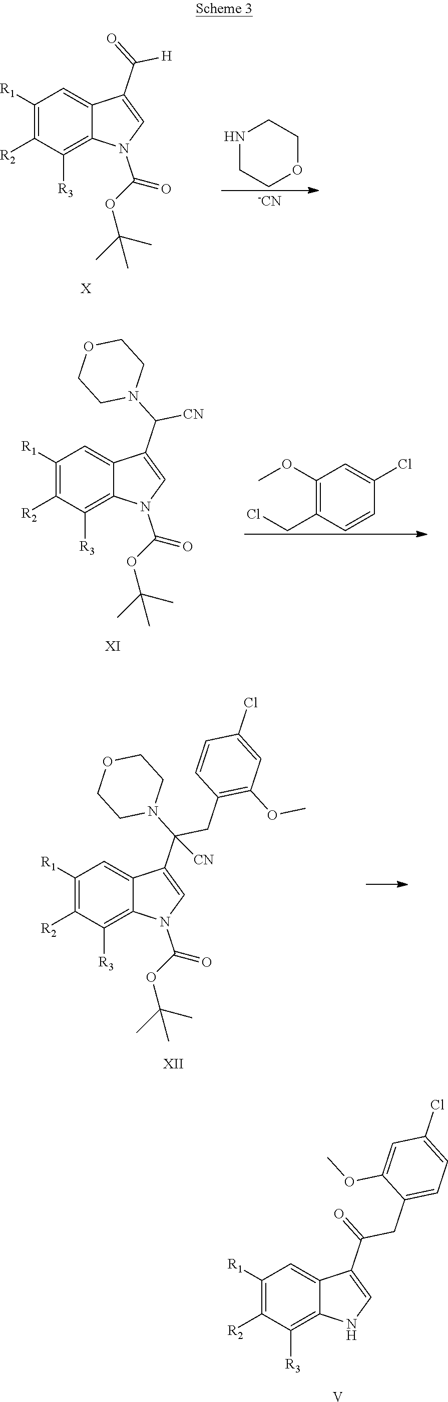

Compounds of Formula (I) can be synthesized according to the procedures disclosed in WO 2016/180696, which is incorporated herein by reference in its entirety.

### EXAMPLES

In the following examples compound (a) is used as active pharmaceutical ingredient (API). Compound (a) was synthesized as described in WO 2016/180696, under Example 9. It was obtained as a white powder:

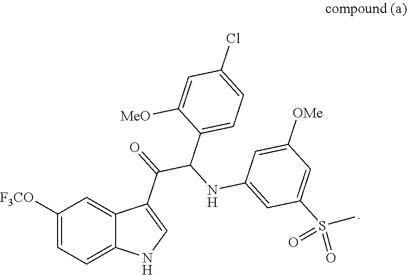 [SMILES: COc1cc(NC(C(=O)c2c[nH]c3ccc(OC(F)(F)F)cc23)c2ccc(Cl)cc2OC)cc(S(C)(=O)=O)c1]

### Reagents

| Name | Grade | Company |
|---|---|---|
| Gelucire 50/13 | N/A | GATTEFOSSÉ |
| PEG3350 | N/A | Colorcon |
| Acetonitrile (ACN) | UPLC | Merck |
| Size 0# HPMC Swedish white | N/A | Capsugel |
| Vitamin-E | N/A | Spectrum chemical |
| Propyl gallate | HPLC | SIGMA |
| Compritol 888 (Glyceryl | N/A | GATTEFOSSÉ |
| dibehenate) | | |
| Geleol Mono and diglycerides | N/A | GATTEFOSSÉ |
| (Glycerol monostearate) | | |
| Cetyl alcohol (C16) | N/A | BASF |
| D-α-tocopherol polyethylene | N/A | N/A |
| glycol 1000 succinate (TPGS) | | |
| Precirol ATO 5 (glyceryl | N/A | GATTEFOSSÉ |
| palmitostearate) | | |
| Tocopherol | N/A | SIGMA |
| L-Ascorbyl 6-palmitate | N/A | Sa-en Chemical |
| (Ascorbic palmitate) | | Scientific, INC |
| PEG1000 | | BASF |
| PEG6000 | | BASF |
| PEG2000 | | BASF |
| PVP VA64 | | BASF |

### Ultra Performance Liquid Chromatography (UPLC) Method

### Column: WATERS ACQUITY UPLC HSS T3 (PDS-HPLC-204/213)

Column length: 150 mm
Column diameter: 2.1 mm
Particle size: 1.8 μm

### Column Temperature 25° C.

Flow rate 0.2 mL/min

### Wavelength 235 nm

Injection volume 2 μL
Mobile Phase A 10 mM CH₃COONH₄ in Water/Acetonitrile (ACN) (95/5)

### Mobile Phase B ACN

Gradient program:

| Time (min) | Mobile Phase A (%) | Mobile Phase B (%) |
|---|---|---|
| 0 | 100 | 0 |
| 18 | 5 | 95 |
| 24 | 5 | 95 |
| 25 | 100 | 0 |
| 30 | 100 | 0 |

Sample Concentration 0.2 mg/mL
Diluent ACN/Water, 50/50, v/v; MeOH:H₂O, 80/20 v/v

### X-Ray Powder Diffractometer Method

X-ray powder diffraction (XRPD) test was carried out on a Bruker D8 Advance X-ray powder diffractometer. The sample was spread on a mono-crystalline silicon plate and using weighing paper and a slight pressure to obtain a flat and homogeneous surface before testing.

Details of the XRPD method used in the tests are mentioned below:

### Tube: Cu: K-Alpha (λ=1.54056 \acute{Å})

### Generator: Voltage: 40 kV; Current: 40 mA

### Detector: PSD: LynxEye

### Divergence Slit: 0.60 mm; Primary Soller Slit: 2.5°.

### Detector Slit: 10.50 mm; Antiscatter Slit: 7.10 mm

Sec. Soller Slit: 2.5°.
Scan type: Locked Coupled
Scan mode: Continuous Scan
Scan parameter: Scan axis: 2-Theta/ThetaScan
Scope: 3 to 50°; Step size: 0.02°.

### Time/step: 0.12 s

Sample rotation: 60 rpm
Scanning rate: 10°/min
Radiation type: CuKα

### Hot Stage Microscope (HSM) Method

Hot stage microscope (HSM) was carried out on Nikon LV100PL polarized light microscope equipped with 5 megapixel CCD. A small amount of sample was dispersed on slide and covered by thin cover glass to eliminate any pollution. Appropriate physical lens was chosen for morphology observation.

Details of the PLM method used in the tests are mentioned below:
Ocular lens: 10×
Physical lens: 10×

### Example 1: Solubility Test in Gelucire 50/13 and PEG 3350

Compound (a) was weighed into a 1.5 mL HPLC vial, added about 1 mL or 880 mg of molten media, then kept stirring at different temperature at 700 rpm (see details in Table 1). Compound (a) was added until a suspension was visually observable. After stirring for about 24 hours, the obtained mixtures were quickly transferred to a filter centrifugal tube and centrifuged at 40° C. for 1 minute at 14000 rpm rate. The solution in the bottom of the filter centrifugal tube was weighed into 10 mL or 25 mL volumetric flasks (about 15-20 mg), diluents were added (ACN: water=1:1 v/v for PEG3350 or MeOH:water=8:2 v/v for Gelucire 50/13), ultrasonicated for 10 min and more diluent was added to scale and analyzed by UPLC (see method above). As shown in Table 1, the solubility of compound (a) in Gelucire 50/13 was slightly higher than 120 mg/g after stirring at 55° C. and 60° C. for 24 hours, while was equal or a little lower than 120 mg/g in PEG 3350 at both at 60° C. and 65° C.

TABLE 1
Solubility of compound (a) in Gelucire 50/13 and PEG 3350

| | | Stirring | | Solubility |
|---|---|---|---|---|
| Vehicle | Temp. | condition | Appearance | (mg/g) |
| Gelucire | 55° C. | 700 rpm for | Light yellow | 141 |
| 50/13 | 60° C. | 24 hours | viscous | 148 |
| PEG 3350 | 60° C. | | suspension | 112 |
| | 65° C. | | | 121 |

### Example 2: Solubility Test in PEG1000

About 0.5 g of compound (a) was weighed into 4 mL glass vials, 1.5 g of molten PEG1000 were then added and kept stirring at 40° C. or 50° C. at 700 rpm. After stirring for 24 hours, the obtained mixtures were transferred into a filter centrifugal tube and centrifuged at 40° C. for 1 minute at 14000 rpm rate. The solution in the bottom of filter centrifugal tube wad weighed into 25 mL volumetric flasks (about 25 mg), diluent was added ACN:H₂O=1:1 v/v, ultrasonicated for 10 min and more diluent was added to scale and analyzed by UPLC (see method above). As shown in Table 2, the solubility of compound (a) in PEG1000 was about 140 mg/g.

TABLE 2
Solubility of compound (a) in PEG 1000

| | | Stirring | | Solubility |
|---|---|---|---|---|
| Vehicle | Temp. | condition | Appearance | (mg/g) |
| PEG 1000 | 40° C. | 700 rpm | Milk white | 141 |
| | 50° C. | for 24 | suspension | 146 |
| | | hours | | |

### Example 3: Solubility Test in TPGS with Different Ratios of PEG1000

About 0.2 g of compound (a) was weighed into 1.5 mL HPLC vials, 0.6 g of molten TPGS, TPGS with 5%, 10% or 15% PEG1000 (in w/w of TPGS) were then added and kept stirring at 45° C. at 700 rpm. Additionally to each of these mixtures, 100 ppm of Ascorbic Palmitate were added. After stirring for 24 hours, they were transferred into filter centrifugal tube and centrifuged at 40° C. for 5 minutes at 14000 rpm rate. The solutions in the bottom of filter centrifugal tube were weighed into 25 mL volumetric flasks (about 10-20 mg), diluent was added ACN:H2O=1:1 v/v, ultrasonicated for 10 min and more diluent was added to scale and analyze by UPLC (see method above). The solid residues after solubility test were collected and analyzed by XRPD (the solid residue in TPGS and TPGS with 5% PEG1000 were washed with water at 50° C. 3 times and analyzed by XRPD). As shown in Table 3, the solubility of compound (a) in TPGS was 274 mg/g, while the solubility decreased as the content of PEG1000 was increased, therefore the PEG1000 would lower the solubility of compound (a) in TPGS.

Solubility test of compound (a) in TPGS with different ratio of PEG1000 after stirring at 45° C. for 96 hours were further tested to determine the effect of time on solubility and XRPD pattern. As shown in Table 3, the solubility of compound (a) in TPGS was 225 mg/g after 96 hours was lower than at 24 hours; however, the solubility further decreased when the content of PEG1000 was increased.

TABLE 3
Solubility of compound (a) in TPGS with different ratio of PEG1000

| | | Time | Solubility | |
|---|---|---|---|---|
| Vehicle | Temp.(° C.) | (h) | (mg/g) | Appearance |
| TPGS | 45 | 24 | 274 | Semitransparent |
| TPGS + 5 vol % | | | 263 | suspension |
| PEG1000 | | | | |
| TPGS + 10 vol % | | | 206 | Milk white |
| PEG1000 | | | | suspension |
| TPGS + 15 vol % | | | 179 | |
| PEG1000 | | | | |
| TPGS + 100 ppm AP | | 96 | 225 | Milk white |
| TPGS + 5 vol % | | | 186 | suspension |
| PEG1000 + 100 ppm AP | | | | |
| TPGS + 10 vol % | | | 157 | |
| PEG1000 + 100 ppm AP | | | | |
| TPGS + 15 vol % | | | 144 | |
| PEG1000 + 100 ppm AP | | | | |

### Example 5: Semi-Solid Formulation Evaluation in TPGS System

### Physical and Kinetic Stability of Mixture of Vehicle and TPGS

Formulations with different excipients were prepared as follows: first TPGS was weighed into 4 mL glass vial and melted at 50° C. Second, one of the following excipients was added in the proportions indicated in Table 4: Compritol 888 (MP 65-77° C.), Cetyl alcohol (MP 46-52° C.), Geleol Mono- and di-glycerides (MP 54-64° C.), Precirol ATO 5 (glyceryl palmitostearate) (MP 50-60° C.), PEG 6000 (MP 55-60° C.) and PEG 3350 (MP 60-65° C.). The resulting mixtures were stirred at 60° C.-70° C. to dissolve, then kept stirring at lower temperature (50° C.-65° C.) to observe appearance of mixed vehicle.

As shown at Table 4, the mixture of TPGS and Compritol 888 could not keep clear below 70° C. Cetyl alcohol and Geleol Mono and diglycerides could be mixed with TPGS well and kept clear at 55° C.

TABLE 4
Preparation of mixed vehicle of TPGS

| | | Dissolution | Time | Keep | Keep | Keep | Keep |
|---|---|---|---|---|---|---|---|
| Excipient | Ratio | Temp. | to | stirring at | stirring at | stirring at | stirring at |
| Mixed | w/w | (° C.) | clear | 50° C. | 55° C. | 60° C. | 65° C. |
| TPGS:Compritol | 90:10 | 70 | Not miscible | / | / | / | / |
| 888 (glyceryl | 95:5 | | <1 h | cloudy | cloudy | cloudy | cloudy |
| dibehenate) | 98:2 | | <1 h | cloudy | cloudy | cloudy | cloudy |
| TPGS:Cetyl | 90:10 | 60 | <1 h | clear | clear | / | / |
| alcohol (C16) | 95:5 | | <1 h | clear | clear | / | / |
| | 98:2 | | <1 h | clear | clear | / | / |
| TPGS: Geleol | 90:10 | 60 | <1 h | cloudy | clear | / | / |
| (Glycerol | 95:5 | | <1 h | clear | clear | / | / |
| mono- and di- | 98:2 | | <1 h | clear | clear | / | / |
| glycerides | | | | | | | |
| monostearate) | | | | | | | |
| TPGS:Precirol | 95:5 | 65 | <1 h | / | clear | / | |
| ATO 5 | 98:2 | | <1 h | / | cloudy | clear | / |
| | 90:10 | | <1 h | / | cloudy | / | / |
| TPGS:PEG6000 | 95:5 | 65 | <1 h | / | clear | / | / |
| | 98:2 | | <1 h | / | clear | / | / |
| | 90:10 | | <1 h | / | clear | / | / |
| TPGS:PEG3350 | 90:10 | 65 | <1 h | / | clear | / | / |

“/”: not performed

Surprisingly, formulations comprising cetyl alcohol and Geleol Mono- and di-glycerides were found to improve the physical stability of TPGS formulation.

Formulations comprising different proportions of Cetyl alcohol and Geleol Mono- and di-glycerides with TPGS mixed vehicle were prepared as described above, and filled into size 0 HPMC Swedish white capsule from Capsugel. The capsules were then stored at 40° C. to observe appearance.

Since the capsules filled with Cetyl alcohol and Geleol Mono- and di-glycerides with TPGS mixed vehicle did not show good stability, other high melting point excipients (Precirol ATO 5, PEG 6000 and PEG 3350) were mixed with TPGS and poured into capsules. The capsules were treated to assess their stability as described above. The results are also shown in Table 5. The results showed that Precirol ATO 5, PEG 6000 and PEG 3350 could be mixed well with TPGS, the mixtures kept clear at 55° C. or 60° C. and kept solid at 40° C.

TABLE 5
Appearance of TPGS mixed vehicles in HPMC capsule at 40° C.

| | | | | | Appearance |
|---|---|---|---|---|---|
| | Ratio | | | Appearance of | of semisolid |
| Sample name | w/w | Time | Temp. | capsules | in vials |
| TPGS:Cetyl | 90:10 | 1 day | 40° C. | Red capsule filled | Clear liquid |
| alcohol | 95:5 | | | with mobile liquid, | |
| | 98:2 | | | no leakage | |
| TPGS:Geleol | 90:10 | | | Red capsule filled | Soft solid, no |
| Mono- and di- | | | | with soft solid, | liquidity |
| glycerides | | | | no liquidity | |
| | 95:5 | | | | Cloudy liquid |
| | 98:2 | | | Red capsule filled | |
| | | | | with mobile liquid, | |
| | | | | no leakage | |
| TPGS:Precirol | 95:5 | 3 days | | Leak | Cloudy liquid |
| ATO 5 | 98:2 | | | Red capsule filled | Slight liquidity |
| | | | | with soft solid, | |
| | | | | no leakage | |
| | 90:10 | | | Red capsule filled | Soft solid, |
| | | | | with soft solid, | no liquidity |
| | | | | no liquidity | |
| TPGS:PEG6000 | 95:5 | | | Red capsule filled | Soft solid, |
| | 98:2 | | | with soft solid, | no liquidity |
| | 90:10 | | | no liquidity | |
| TPGS:PEG3350 | 90:10 | | | Red capsule filled | Soft solid, |
| | | | | with soft solid, | no liquidity |
| | | | | no liquidity | |

The solubility of these mixed excipients with compound (a) was then tested. Different ratios of the excipients were mixed at different temperatures (Table 6); then compound (a) was added and the mixtures stirred at the same temperature. The samples were analyzed by UPLC (see method above). The solubility results are depicted in Table 6; these results show that compound (a) dissolves better in the Precirol ATO 5/TPGS system (>200 mg/g).

TABLE 6
Solubility of compound (a) in mixed excipients

| | Ratio | Temp. | Solubility | |
|---|---|---|---|---|
| Media | w/w | (° C.) | (mg/g) | Appearance |
| TPGS:ATO5 | 95:5 | 60 | 270.0 | Almost clear solution |
| | 98:2 | | 228.2 | Milk white suspension |
| | 90:10 | | 216.1 | Milk white suspension |
| | 95:5 | 65 | 295.5 | Almost clear solution |
| | 98:2 | | 248.5 | Milk white suspension |
| | 90:10 | | 219.0 | Milk white suspension |
| TPGS:PEG6000 | 95:5 | 55 | 81.0 | Milk white suspension |
| | 98:2 | | 63.0 | Milk white suspension |
| | 90:10 | | 60.8 | Milk white suspension |
| | 95:5 | 60 | 84.0 | Milk white suspension |
| | 98:2 | | 66.8 | Milk white suspension |
| | 90:10 | | 66.6 | Milk white suspension |
| TPGS:PEG3350 | 90:10 | 55 | 71.0 | Milk white suspension |
| | 90:10 | 60 | 76.2 | Milk white suspension |

### Physical and Kinetic Stability of Semi-Solid Formulations of Compound (a)

To assess the physical stability of the formulations, a 120 mg/g semi-solid formulation of compound (a) in TPGS: Precirol ATO 5=90:10 w/w (Formulation 1) was prepared by first mixing the excipients at 60° C. and then adding compound (a) and stirring the formulation for 40 minutes at 60° C. to dissolve the compound.

Size 0 HPMC capsules were filled with Formulation 1 to conduct leak test in 40° C. drying oven. Additionally, Formulation 1 was poured into 4 pieces in 4 mL glass vials and stored at different conditions (5° C.-closed, 25° C./60% Relative Humidity (RH)-closed, 30° C./65% RH-closed and 40° C./75% RH-closed) to observe physical stability by Hot Stage Microscope (HSM) (see method above). Samples were also analyzed by UPLC. The results are summarized in Table 7.

After being stored at 40° C. for up to 14 days, Formulation 1 in vial was still a soft solid without liquidity. The capsules showed no leakage. The HSM of Formulation 1 showed that the formulation was stable after storing at different conditions (5° C.-closed, 25° C./60% RH-closed, 30° C./65% RH-closed and 40° C./75% RH-closed) for 14 days.

TABLE 7
Appearance of formulations of compound
(a) according to the invention

| | | | | | Appearance |
|---|---|---|---|---|---|
| | | | | Appearance | of semisolid |
| Sample name | Time | | Temp. | of capsules | in vials |
| Formulation 1 | 3 | days | 40° C. | Red capsule | Soft solid, |
| | 7 | days | | filled with | no liquidity |
| | 10 | days | | soft solid, | |
| | 40 | days | | no leakage | |
| | ~40 | days | | | |

To assess the kinetic stability of the formulations according to the invention, two formulations were prepared:

Formulation 2: 120 mg/g compound (a) in TPGS: Precirol ATO 5=90:10 w/w.
Formulation 3: 120 mg/g compound (a) in TPGS: ATO 5=90:10 w/w with 0.2 wt % Tocopherol and 0.1 wt % Ascorbic Palmitate.

The formulations were prepared by first mixing TPGS: Precirol ATO 5=90:10 w/w and the eventual antioxidants at 60° C., then adding compound (a) and stirring at 60° C. for about 40 mins to obtain formulations. About 33.3 mg of the formulations was weighed into 40 mL glass vials at initial, 1 h, 3 h, 5 h, 7 h, 24 h, 48 h and 72 h, then diluent (ACN:water=1:1, v/v) was added and ultrasonicated for 10 min, then filtered through filter and analyzed by UPLC.

After stirring for up to 3 days at 60° C., the total impurity (%) of Formulation 2 increased by ˜0.35% while the total impurity (%) of Formulation 3 had no significant change (see Table 8), indicating that Formulation 3 was stable at 60° C. for 3 days.

TABLE 8
Kinetic stability of formulations of compound
(a) according to the invention

| | | | | Label | Total |
|---|---|---|---|---|---|
| | | Time | | claim | impurity |
| Sample name | Temperature | point | | (%) | (%) |
| Formulation 2 | Stirred at | Initial | | 100.1 | 0.91 |
| | 60° C. for 40 | 1 | h | 101.3 | 0.92 |
| | mins to dissolve, | 3 | h | 100.5 | 0.93 |
| | then keep stirring | 5 | h | 100.2 | 0.92 |
| | at 60° C. for | 7 | h | 99.6 | 0.93 |
| | another 3 days | 24 | h | 99.1 | 1.02 |
| | | 48 | h | 99.6 | 1.12 |
| | | 72 | h | 98.7 | 1.23 |

| | | | |
|---|---|---|---|
| Formulation 3 | Initial | 100.5 | 0.90 |

| | | | |
|---|---|---|---|
| 1 | h | 101.2 | 0.89 |
| 3 | h | 101.7 | 0.89 |
| 5 | h | 102.1 | 0.89 |
| 7 | h | 101.3 | 0.89 |
| 24 | h | 101.4 | 0.89 |
| 48 | h | 99.9 | 0.89 |
| 72 | h | 101.5 | 0.89 |

Further investigation of the kinetic stability of formulations according to the invention with low content of antioxidants were conducted. Three Formulations were tested:

Formulation 4: TPGS: Precirol ATO 5=90:10 w/w with 200 ppm Tocopherol and 100 ppm Ascorbic Palmitate
Formulation 5: TPGS: Precirol ATO 5=90:10 w/w with 200 ppm Tocopherol
Formulation 6: TPGS: Precirol ATO 5=90:10 w/w with 100 ppm Ascorbic Palmitate

In all three formulations, the TPGS, Precirol ATO 5 and antioxidant were mixed at 60° C. Then compound (a) was added and stirred at 60° C. for about 40 mins to dissolve. The formulations were clear. Then the formulations were kept under stirring for 3 days. Samples were taken at different time points and were analyzed by UPLC. The results are summarized in Table 9. After stirring for up to 3 days at 60° C., the total impurity (wt %) of Formulation 5 increased by ˜0.1 wt % while the total impurity (wt %) of Formulations 4 and 6 had no significant change.

TABLE 9
Kinetic stability of compound (a) formulations
according to the invention

| | | | | Label | Total |
|---|---|---|---|---|---|
| | | Time | | claim | impurity |
| Sample name | Temperature | point | | (wt %) | (wt %) |
| Formulation 4 | Stirred at | Initial | | 99.9 | 0.89 |
| | 60° C. for 40 | 1 | h | 100.7 | 0.90 |
| | mins to dissolve, | 3 | h | 100.5 | 0.90 |
| | then keep stirring | 5 | h | 101.3 | 0.90 |
| | at 60° C. for | 7 | h | 102.2 | 0.89 |
| | another 3 days | 24 | h | 100.8 | 0.89 |
| | | 48 | h | 100.7 | 0.89 |
| | | 72 | h | 100.6 | 0.90 |

| | | | |
|---|---|---|---|
| Formulation 5 | Initial | 99.7 | 0.89 |

| | | | |
|---|---|---|---|
| 1 | h | 100.9 | 0.91 |
| 3 | h | 100.7 | 0.90 |
| 5 | h | 100.7 | 0.90 |
| 7 | h | 101.6 | 0.90 |
| 24 | h | 100.2 | 0.94 |
| 48 | h | 100.7 | 0.99 |
| 72 | h | 100.4 | 1.02 |

| | | | |
|---|---|---|---|
| Formulation 6 | Initial | 100.4 | 0.90 |

| | | | |
|---|---|---|---|
| 1 | h | 100.3 | 0.89 |
| 3 | h | 101.0 | 0.90 |
| 5 | h | 101.2 | 0.90 |
| 7 | h | 102.7 | 0.90 |
| 24 | h | 99.7 | 0.91 |
| 48 | h | 104.0 | 0.91 |
| 72 | h | 100.4 | 0.89 |

### Example 6, Comparative Example: Semi-Solid Formulation Evaluation in PEG2000 System

The kinetic and physical stability of formulations of compound (a) in PEG2000 with different excipients was evaluated. The following formulations were prepared:

Comparative formulation 1: 120 mg/g compound (a) in PEG2000.
Comparative formulation 2: 120 mg/g compound (a) in PEG2000 with 5 wt % copolymer of N-vinylpyrrolidone and vinyl acetate 64 (PVP VA64).
Comparative formulation 3: 120 mg/g compound (a) in PEG2000 with 5 wt % PVP VA64 and 0.2 wt % Propyl Gallate.

The formulations were prepared by warming up the excipients at 60° C., then adding compound (a) and stirring at 60° C. for about 2.5 hours to obtain clear formulation.

The formulations were poured into 4 glass vials of 4 mL capacity and stored at different conditions (5° C.-closed, 25° C./60% Relative Humidity (RH)-closed, 30° C./65% RH-closed and 40° C./75% RH-closed) to observe physical stability by Hot Stage Microscope (HSM) (see method above). The results are summarized in Table 10.

The HSM of Comparative formulation 1 showed that the formulation was unstable after storing at all conditions (5° C.-closed, 25° C./60% RH-closed, 30° C./65% RH-closed and 40° C./75% RH-closed) for 3 days. Comparative formulation 3 was unstable at 40° C./75% RH-closed for 3 days. The HSM of Comparative formulation 2 showed that the formulation was stable after storing at different conditions (5° C.-closed, 25° C./60% RH-closed, 30° C./65% RH-closed and 40° C./75% RH-closed) for 3 days. However, birefringent crystals occurred in HSM after the formulation was stored at 40° C./75% RH-closed for 6 days and white particles appeared in the formulation after heating to 60° C. Comparative formulation 2 was stable after storing at 5° C.-closed, 25° C./60% RH-closed and 30° C./65% RH closed for 6 days (results not shown).

TABLE 10
Physical stability of compound (a) comparative formulation

| Formulation | Conditions | Time point | Appearance |
|---|---|---|---|
| Comparative | 5° C. closed | 3 days | Crash out |
| formulation 1 | 25° C./60% RH closed | | |
| | 30° C./65% RH closed | | |
| | 40° C./75% RH closed | | |
| Comparative | 5° C. closed | 3 days | No crash out |
| formulation 2 | | 6 days | No crash out |
| | 25° C./60% RH closed | 3 days | No crash out |
| | | 6 days | No crash out |
| | 30° C./65% RH closed | 3 days | No crash out |
| | | 6 days | No crash out |
| | 40° C./75% RH closed | 3 days | No crash out |
| | | 6 days | Crash out |
| Comparative | 5° C. closed | 3 days | No crash out |
| formulation 3 | 25° C./60% RH closed | 3 days | No crash out |
| | 30° C./65% RH closed | 3 days | No crash out |
| | 40° C./75% RH closed | 3 days | Crash out |

Comparative formulations 2 and 3 were then subjected to a kinetic stability test. About 33.3 mg of comparative formulations were weighed into 40 mL glass vials at initial, 2 h, 4 h and 24 h, then diluent (ACN:water=1:1, v/v) was added and ultrasonicated for 10 min. The samples were analyzed by UPLC.

After stirring for 24 hours at 55° C., the total impurity (%) of both comparative formulations had increased (Table 11). Propyl Gallate has no anti-oxidation effect in Comparative formulation 3, on the contrary the total impurity (%) was little higher in Comparative formulation 3 than in Comparative formulation 2.

TABLE 11
Kinetic stability of Comparative formulations
of compound (a) at 55° C.

| | | | Label | Total |
|---|---|---|---|---|
| | | Time | claim | impurity |
| Sample name | Temperature | point | (%) | (s %) |
| Comparative | Stirred at | Initial | 100.7 | 1.01 |
| formulation 2 | 60° C. for 2.5 | 2 h | 99.7 | 1.03 |
| | hours to | 4 h | 99.9 | 1.06 |
| | dissolve, then | 24 h | 98.3 | 1.34 |
| Comparative | keep stirring | Initial | 99.9 | 1.02 |
| formulation 3 | at 55° C. for 24 | 2 h | 101.3 | 1.06 |
| | hours | 4 h | 100.8 | 1.07 |
| | | 24 h | 99.7 | 1.41 |

### Preclinical Study

A pharmacokinetic (PK) study was carried out using the following formulations:
Comparative formulation: compound (a), TPGS in Hypromellose (HPMC) in capsules,
Formulation according to the invention: compound (a), TPGS/ATO 90:10 w/w in capsules.

Compound (a) was formulated in 50 mg capsules for assessment in a fasted dog PK study. A single dose was administered to fasted male beagle dogs (N=3/group) with at least a 7-day washout period between doses. The comparative formulation was administered to two separate groups of dogs while the formulation according to the invention was administered to a one group of dogs.

TABLE 12
Fasted dog pharmacokinetics

| | | | Formulation |
|---|---|---|---|
| | Comparative | Comparative | according to the |
| | formulation I | formulation I | invention |
| N | 3 | 3 | 3 |
| Cₘₐₓ | 1050 ± 196 | 970 ± 144 | 1040 ± 465 |
| (ng/mL) | | | |
| Tₘₐₓ | 4.00 (4.00-4.00) | 4.00 (2.00-4.00) | 4.00 (4.00-4.00) |
| AUCₗₐₛₜ | 13000 ± 1770 | 11300 ± 1430 | 14300 ± 4590 |
| (ng + h/mL) | | | |
| AUC_{∞} | 13800 ± 2170 | 11700 ± 1660 | 14600 ± 4400 |
| (ng + h/mL) | | | |

Mean ± standard deviation
AUC = area under the plasma concentration-time curve; AUCₗₐₛₜ = AUC calculated until the last timepoint of blood plasma level; AUC_{∞} = AUC calculated using extrapolation of the plasma profile to infinity time; Cₘₐₓ = maximum observed plasma concentration; N = number of animals; Tₘₐₓ = time correspondent to the maximum observed plasma concentration.

TABLE 13
Dog Pharmacokinetics at different doses

Formulation according to the invention

| Dose | 50 mg/dog | 2*50 mg/dog |
|---|---|---|
| N | 3 | 3 |
| Cₘₐₓ (ng/mL) | 1040 ± 465 | 1170 ± 392 |
| Tₘₐₓ | 4.00 (4.00-4.00) | 4.00 (4.00-4.00) |
| AUCₗₐₛₜ (ng + h/mL) | 14300 ± 4590 | 21800 ± 12000 |
| AUC_{∞} (ng + h/mL) | 14600 ± 4400 | 22000 ± 12200 |

Mean ± standard deviation

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention and that embodiments within the scope of these claims and their equivalents be covered thereby.

1. A pharmaceutical formulation, comprising:
a) D-α-tocopherol polyethylene glycol succinate (TPGS)
b) an active pharmaceutical ingredient that is soluble in molten TPGS; and
c) glyceryl palmitostearate.

2. The pharmaceutical formulation of claim 1, wherein the pharmaceutical formulation is a semi-solid formulation.

3. The pharmaceutical formulation of claim 1, wherein the TPGS and the glyceryl palmitostearate are present in said pharmaceutical formulation in a ratio of from 95:5 w/w to 70:30 w/w.

4. The pharmaceutical formulation of claim 1, wherein the pharmaceutical formulation further comprises an antioxidant.

5. The pharmaceutical formulation of claim 4, wherein the pharmaceutical formulation comprises from 0.001 wt % to 2.0 wt % of the antioxidant relative to the total weight of the formulation.

6. The pharmaceutical formulation of claim 4, wherein the antioxidant is selected from ascorbic palmitate, tocopherol (vitamin E), thiodipropionic acid, lipoic acid, hydroquinone, phytic acid, monothioglycerol, sodium thioglycolate, thioglycol, beta carotene, butylated hydroxyanisole (BHA), butylated hydroxytoluene (BHT), cysteine, cysteine hydrochloride, propyl gallate (PG), sodium metabisulfite, ascorbyl stearate, potassium metabisulfite, disodium EDTA (ethylenediamine tetraacetic acid; also known as disodium edentate), EDTA, erythorbic acid, ethoxyquin, glutathione, gum guaiac, lecithin, propyl gallate, TBHQ (tert butyl hydroxyquinone), tartaric acid, citric acid, citric acid monohydrate, methane sulfonic acid, methionine, sodium metabisulfite, sodium thiosulfate, sodium sulphite, and combinations thereof.

7. The pharmaceutical formulation of claim 1, wherein the pharmaceutical formulation comprises from 0.1 wt % to 40 wt % of the active pharmaceutical ingredient relative to the total weight of the pharmaceutical formulation.

8. The pharmaceutical formulation of claim 1, wherein the active pharmaceutical ingredient is a dengue viral replication inhibitor.

9. The pharmaceutical formulation of claim 1, wherein the active pharmaceutical ingredient is a compound of Formula (I) 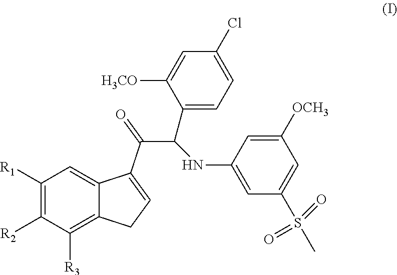
a stereo-isomeric form, a pharmaceutically acceptable salt, solvate or polymorph thereof said compound is selected from the group wherein:
R₁ is H, R₂ is F and R₃ is H or CH₃,
R₁ is H, CH₃ or F, R₂ is OCH₃ and R₃ is H and
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H and
R₁ is OCF₃, R₂ is H and R₃ is CH₃.

10. The pharmaceutical formulation according to claim 9, wherein the compound of Formula (I) is: 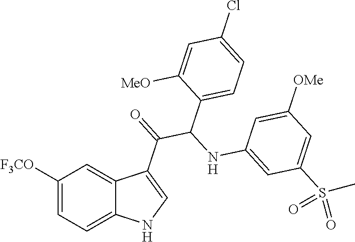 [SMILES: COc1cc(NC(C(=O)c2c[nH]c3ccc(OC(F)(F)F)cc23)c2ccc(Cl)cc2OC)cc(S(C)(=O)=O)c1]
or a stereo-isomeric form, a pharmaceutically acceptable salt, solvate or polymorph thereof.

11. A solid dosage form comprising the pharmaceutical formulation of claim 1.

12. The solid dosage form of claim 11, wherein the pharmaceutical formulation comprises from 0.5 mg to 1000 mg of the active pharmaceutical ingredient.

13. A method for treating or preventing dengue viral infections comprising administering to a subject in need thereof the pharmaceutical formulation of claim 1.

14. A process for preparing a pharmaceutical formulation according to claim 1, comprising the steps of:
a) forming a melt comprising glyceryl palmitostearate and D-α-tocopherol polyethylene glycol 1000 succinate (TPGS), wherein the step of forming a melt comprises heating polyethylene glycol 1000 succinate (TPGS) to a temperature above its melting point; and
b) mixing the active pharmaceutical ingredient and stirring at the temperature of step a) until said active pharmaceutical ingredient dissolves;
to provide a pharmaceutical formulation according to claim 1.

15. The process of claim 14, wherein the active pharmaceutical ingredient is a compound of Formula (I) 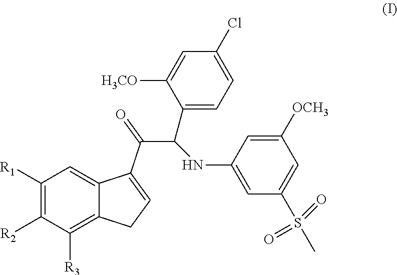
a stereo-isomeric form, a pharmaceutically acceptable salt, solvate or polymorph thereof; said compound is selected from the group wherein:
R₁ is H, R₂ is F and R₃ is H or CH₃,
R₁ is H, CH₃ or F, R₂ is OCH₃ and R₃ is H and
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H and
R₁ is OCF₃, R₂ is H and R₃ is CH₃.

16. The pharmaceutical formulation of claim 1, wherein component a) is D-α-tocopherol polyethylene glycol 1000 succinate.

17. The pharmaceutical formulation of claim 5, wherein the pharmaceutical formulation comprises from 0.005 wt % to 1.0 wt % of the antioxidant.

18. The pharmaceutical formulation of claim 17, wherein the pharmaceutical formulation comprises from 0.005 wt % to 0.5 wt % of the antioxidant.

19. The pharmaceutical formulation of claim 7, wherein the pharmaceutical formulation comprises from 1 wt % to 30 wt % of the active pharmaceutical ingredient relative to the total weight of the pharmaceutical formulation.

20. The pharmaceutical formulation of claim 19, wherein the pharmaceutical formulation comprises from 5 wt % to 25 wt % of the active pharmaceutical ingredient relative to the total weight of the pharmaceutical formulation.

21. The solid dosage form of claim 12, wherein the pharmaceutical formulation comprises from 1 mg to 1000 mg of the active pharmaceutical ingredient.

22. The solid dosage form of claim 21, wherein the pharmaceutical formulation comprises from 2 mg to 500 mg of the active pharmaceutical ingredient.

23. The process of claim 15, wherein the active pharmaceutical ingredient is 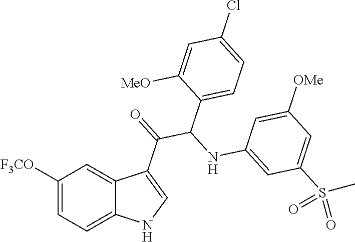 [SMILES: COc1cc(NC(C(=O)c2c[nH]c3ccc(OC(F)(F)F)cc23)c2ccc(Cl)cc2OC)cc(S(C)(=O)=O)c1]
or a stereo-isomeric form, a pharmaceutically acceptable salt, solvate or polymorph thereof.
